(19) United States
(12) Patent Application Publication
(10) Pub. No.: US 2015/0307924 A1
(43) Pub. Date: Oct. 29, 2015

(54) METHODS FOR DIAGNOSING IRRITABLE BOWEL SYNDROME

(71) Applicant: AAK PATENT B.V., Willemstad (NL)

(72) Inventors: Lambertus TUK, Willemstad (NL); Willem Meindert DE VOS, Ede (NL); Mirjana RAJILIC-STOJANOVIC, Beograd (RS)

(73) Assignee: AAK PATENT B.V., Willemstad (NL)

(21) Appl. No.: 14/796,652

(22) Filed: Jul. 10, 2015

Related U.S. Application Data
(62) Division of application No. 13/500,194, filed on May 30, 2012, now abandoned.
(60) Provisional application No. 61/248,601, filed on Oct. 5, 2009.

(30) Foreign Application Priority Data
Oct. 5, 2009 (EP) 09172243.9
Dec. 22, 2009 (EP) 09180434.4

(51) Int. Cl. C12Q 1/68

(52) U.S. Cl. CPC C12Q 1/689; C12Q 2600/158

(57) ABSTRACT

The present invention discloses a method for diagnosing Irritable Bowel Syndrome (IBS) in a test sample by determining the level of several bacterial taxa in the test sample, comparing this level with the levels of those bacterial taxa in a control sample, and relating the level to a diagnosis of IBS. Additionally, the present invention provides a method for treatment of IBS based on said diagnosis. Also, the invention provides a method for subtyping IBS in a test sample.

### CROSS REFERENCE TO RELATED APPLICATIONS

This application is a Divisional of U.S. application Ser. No. 13/500,194, filed on May 30, 2012. U.S. application Ser. No. 13/500,194 is the National Phase of PCT/NL2010/050645 filed on Oct. 5, 2010, which claims priority under 35 U.S.C. 119(e) to U.S. Provisional Application No. 61/248,601 filed on Oct. 5, 2009, and under 35 U.S.C. 119(a) to Patent Application Nos. 09172243.9 and 09180434.4 filed in the European Patent Office on Oct. 5, 2009 and Dec. 22, 2009 respectively, all of which are hereby expressly incorporated by reference into the present application.

### FIELD OF THE INVENTION

The present invention is in the field of microbiology and gastrointestinal health, and relates to the use of the gastrointestinal microbiota as a biomarker for intestinal aberrations, notably Irritable Bowel Syndrome.

### BACKGROUND

The gastro-intestinal tract is colonized since birth by complex communities of microbes, including bacteria, archaea and fungi, that develop in time and space. These microbial communities were collectively termed gut microflora in previous times but are now known as gut microbiota that is of a highly complex nature. (Rajilic-Stojanovic et al. 2007. Environ Microbiol 9: 2125-2136) The gut microbiota is involved in a variety of metabolic functions, such as the processing of food components that are not digested by the host, the synthesis of vitamins and the production of short chain fatty acids. However, in recent years it has been established that gut microbes interact with the host cells resulting in modulation of host processes including gut motility, gut barrier and immune function (Zoetendal et al., 2008. Gut 57: 1605-1615). Hence, aberrations in the gut microbiota can be associated with a variety of functional intestinal disorders, including Inflammatory Bowel Disease (hereinafter also referred to as “IBD”) and Irritable Bowel Syndrome (hereinafter also referred to as “IBS”). IBD includes mainly Crohn's Disease and Ulcerative Colitis that are manifested by recurrent severe bouts of inflammation of various parts of the intestinal tract. IBS is a multi-factorial and complex disorder clinically characterized by recurrent episodes of abdominal discomfort or pain, altered bowel habit and urge. Apart from IBD and IBS also other diseases are known to be associated with aberrations in microbiota and these include obesity, the various types of diabetes such as type I diabetes and type II diabetes, Autistic Spectrum Disorder (ASD) related diseases, celiac disease and some forms of cancer (Zoetendal et al, 2008, supra).

From all the diseases that affect the gastro-intestinal tract, IBS is the most prevalent functional bowel disorder, that affects up to 20 percent of the general population in the world. Furthermore, IBS is associated with a high rate of absenteeism from work, a significant impairment in quality of life and substantial health care costs. The diagnosis of IBS is based on aberrant bowel functions using the so called Rome criteria and three subtypes of IBS are discriminated, including the constipation (IBS-C), diarrhea (IBS-D) and alternating constipation/diarrhea (IBS-A) subtypes (Thompson et al., 1989. Gastroenterology 130: 1552-1556; Longstreth et al., 2006. Gastroenterology 130: 1480-1491). While the diagnosis of IBD is based on non-invasive diagnostic procedures as the presence of inflammatory biomarkers in the blood, imaging diagnostics and endoscopic observations (including histology of mucosal specimens), IBS is much harder to diagnose. Nowadays, IBS can only be diagnosed by exclusion of IBD and other bowel disorders (such as celiac disease, colorectal cancer and lactose malabsorption) and is dependent on an anamnesis as laid down in the Rome criteria. This makes the diagnosis of IBS a rather undefined ‘exclusion diagnosis’ and relatively expensive. Hence there is a great need to develop biomarkers that are indicative of IBS, as is confirmed by the US National Institute of Health that states that no test for IBS is known (http://digestive.niddk.nih.gov/ddiseases/pubs/ibs/). Specifically, reliable non-invasive biomarkers are needed to develop a diagnostic test for IBS. These biomarkers can be used to diagnose IBS but also will be instrumental in defining IBS or sub-classifying IBS as well as monitoring the pharmacological responses to a therapeutic intervention. Moreover, the identification of such biomarkers may lead to the discovery and development of new and innovative therapeutic interventions for IBS.

The pathophysiologic pathway of IBS is unknown, and diagnostic procedures, among other by blood analysis, endoscopy, histology and radiologic procedures, do not reveal any common structural abnormalities in the digestive tract. While for a long time IBS has been considered a psychosomatic abberation, in recent years support has been provided for the involvement of biological and hereditary factors concerning the hypersensitivity of the brain-gut axis. Recent studies provide several lines of evidence that support a relation between intestinal microbiota and IBS. In various cases IBS is triggered in previously healthy individuals by acute GI tract infection (gastro-enteritis) by external microbiota resulting in the so called post-infective IBS: up to 25% of patients with acute GI tract infection develop IBS. During these infections the intestinal function and microbiota composition is affected. In several cases successful treatment of IBS has been shown by the consumption of pre- and probiotics that are all known to affect the intestinal microbiota composition and function (Spiller, 2009. Aliment Pharmacol Ther 28: 385-396). Finally, there are observations that IBS subjects in comparison with healthy individuals show deviations in intestinal microbiota composition or metabolites. However, no clear picture emerges from these studies as to what are the specific microbes or microbial groups that differ between IBS and healthy subjects. This is partly caused by the fact that in many cases use is made of culturing techniques to identify microbes, where it is well known that many of the intestinal microbes can not been cultured, and cultivation therefore is known to give significant biases.

US 2008/182291 describes a method of diagnosing constipation in a subject by analysing a breath, flatus, blood or saliva sample from a subject for the presence of methane. Alternatively, a stool sample may be analysed for the presence of at least one methanogenic organism, selected from Ruminococcus sp., Methanobrevibacter sp., Bacteroides sp., Clostridium sp., and Methanobacter sp. However, none of Ruminococcus sp., Bacteroides sp., and Clostridium sp. are methane-producing organisms. Methanobrevibacter sp. and Methanobacter sp. are methane-producing organisms, but they do not belong to the Kingdom Bacteria but rather to the Kingdom Archeae.

Recently, molecular methods have been used in attempts to determine differences between IBS and healthy subjects. Approaches based on quantitative polymerase chain reaction (qPCR) of small parts (usually less than 100 nucleotides) of the 16S rRNA gene gave some indication of differences between a variable set of microbial groups without leading to consistent outcomes. Initial studies were done with limited microbiological and statistical power and showed that in comparison with fecal samples from healthy individuals, IBS subjects contain more Clostridium coccoides and Bifidobacterium catenulatum (Malinen et al., 2005. Am J Gastroenterol. 100:373-82). However, in another study, 6 IBS-C subjects showed a reduced number of bacteria belonging to the Clostridium coccoides/Eubacterium rectale cluster in comparison with healthy controls (Maukonen et al., 2006. J Med Microbiol 55: 625-633). The C. coccoides/E. rectale group is the largest and most dominant bacterial group in the intestinal tract representing up to half of the total microbiota. Hence it can not as such be used in diagnostics as is also indicated by the authors of this study who note that the target C. coccoides-E. rectale group (phylogenetic clusters XIVa and XIVb) is too large to detect subtle variations between the microbiota of control and IBS subjects. Therefore, this group needs to be divided into smaller subgroups in further studies (Maukonen et al., 2006, supra). In a recent study, DNA extracted from pooled fecal samples derived from 23 healthy and 24 subjects with different IBS types was fractionated according to its guanine and cytosine (G+C) content followed by sequence analysis of 16S rDNA clone libraries (Kassinen et al., 2007. Gastroenterology 2007; 133: 24-33). While some differences were observed in 3 of the over 15 fractions, this approach is not quantitative and known to be affected by cloning bias. Moreover, the used approach includes a density gradient centrifugation step to fractionate the DNA samples according to their G+C content that is not applicable for routine diagnostics. However, in the same study also specific qPCRs were performed that showed statistically significant but only slightly larger and highly variable numbers of Collinsella aerofaciens, Clostridium cocleatum-related and Coprococcus eutactus-related bacteria as compared to samples from healthy controls (Kassinen et al., 2007, supra). This study also indicated that differences for other members of Firmicutes remained statistically non-significant. Collinsella aerofaciens belongs to the Actinobacteria, Gram-positive bacteria with a high G+C content. The other two groups are part of the Firmicutes, Gram-positive bacteria with a low G+C content and Clostridium cocleatum-related bacteria constitute a small group in the Clostridium cluster XVIII while Coprococcus eutactus-related bacteria form a minor group in the Clostridium coccoides/Eubacterium rectale (Clostridium cluster XIVa) cluster, including also Eubacterium ruminantium and several not yet cultured phylotypes (see Table 3).

In conclusion, the qPCR approaches provided no clear signature of IBS dysbiosis and it has been stated recently that the results reported so far are conflicting and likely explained by variations in experimental design (Codling et al., Dig Dis Sci 2010 February; 55(2):392-397). Moreover, these conflicting results can also be caused by the heterogeneity of IBS with respect to etiology, pathophysiology and symptomatology. Indeed, in many cases only a limited number of intestinal samples from IBS and healthy subjects is analyzed and in some cases these are derived from the same study (Malinen et al., 2005, supra; Mättö et al., 2005. FEMS Immunol Med Microbiol 43: 213-222; Maukonen et al., 2006, supra; Kassinen et al., 2007, supra). Moreover, in some cases only a specific subtype of IBS is addressed or samples are pooled prior to analysis which precludes analysis of variations. In a recent study specific groups of bacteria were enumerated using fluorescent in situ hybridization (FISH) with specific 16S rRNA gene probes or qPCR analysis of part of the 16S rRNA gene (Kerckhoffs et al., 2009. World J Gastroenterol 2009 Jun. 21; 15(23): 2887-2892). A lower number of Bifidobacteria and no other differences in the major intestinal groups was found in 41 IBS subjects as compared to healthy controls—this included the C. coccoides/E. rectale (Clostridium cluster XIVa) cluster that showed no differences. However, careful analysis of the reported data shows that the lower number of Bifidobacteria was restricted to only the 14 IBS-D subjects and specifically included the Bifidobacterium catenulatum group. These results were corroborated with brush samples from duodenal mucosa, indicating that fecal samples constitute useful material for assessing the state of the microbiota in the gastro-intestinal tract.

The highest number of IBS subjects analysed in a single comparative study reported so far is a recent comparison that included 47 IBS and 33 healthy subjects (Codling et al, 2009, supra). By using a rather qualitative method revealing sequence variations in 16S rRNA genes, ie separating 16S rRNA gene amplicons by Denaturing Gradient Gel Electrophoresis (DGGE), global differences were observed between fecal samples from IBS subjects and healthy controls (Codling et al, 2009, supra). This study supported the possibility to differentiate between IBS and healthy subjects but failed to reveal any specific microbial group or species that could be associated with this difference.

A limited number of studies addressed the dynamics over time of the fecal microbiota in IBS subjects in comparison with that of healthy individuals. A study based on DGGE analysis suggested reduced temporal stability in IBS subjects but used visual inspection and did not correct for the use of antibiotics (Matto et al., 2005, supra). A follow up study with the appropriate corrections for the use of antibiotics showed that for periods of 3 months in 16 IBS subjects compared to 16 matched healthy subjects, the temporal stability of the Clostridium histolyticum group (also known as Clostridium cluster I and II) was higher in the IBS-c type than in the healthy subjects (Maukonen et al. 2006, supra). The methods of DGGE analysis due to their low resolution however lead to inconsistent results and outcomes that are notoriously difficult to reproduce. In addition, only a profile is generated without any link to taxonomic information. Moreover, as these methods can be best applied on small amplicons (around a few hundred bp) they have been only applied in addressing the sequence variation in the V1-V3 region of the 16S rRNA genes. Finally, the methods based on DGGE are laborious, time-consuming and have significant gel to gel variations and require relatively long processing times—hence they can not be used as a routine diagnostic tool. A summary of the drawbacks of the so far used methods is provided in a recent review that also indicates the need for IBS diagnostics and clinical algorithms that would identify subjects with differing causes of IBS as a way to improve the results of therapies, varying from pharmaceutical treatments to dietary, probiotics and prebiotics interventions (Parkes et al., 2008. Am J Gastroenterol 2008; 103:1557-1567).

Recently, a human-intestine specific phylogenetic microarray has been developed and validated that provides a way to provide high throughput data of the intestinal microbiota in an accurate way over a large dynamic range (Zoetendal et al., 2008, supra; Rajilic-Stojanovic et al., 2009. Environ Microbiol 11: 1736-1743). In a preliminary study using a first version of the HITChip, 20 IBS and 20 healthy subjects were compared—apart from an increased level of Bacillus spp and reduced level of Bacteroides spp in IBS subjects that could not be specified, no other significant differences were observed between IBS and healthy subjects (M. Rajilic-Stojanovic, Diversity of the human gastro-intestinal microbiota, PhD thesis Wageningen University 2007, pp 116-134). This can be attributed to a limited number of subjects and use of a first version of the HITChip with redundant probes. In this study only significant differences between healthy subjects and subjects with subtypes of IBS, i.e. IBS-A, IBS-C, IBS-D, were observed for some bacterial groups. This limits any clinical application as a general diagnostic tool for IBS.

Hence, there is a need in the art to identify biomarkers that are indicative of IBS, preferably non-invasive biomarkers, that can be used to develop a diagnostic test for IBS. Moreover, such biomarkers indicative of IBS may be instrumental in defining IBS and/or subtyping IBS, as well in monitoring pharmaceutical responses to a therapeutic intervention. Moreover, such biomarkers may allow discovery and development of new and innovative therapeutic interventions for IBS.

### FIGURES

The invention will be illustrated using the appended Figure, in which:

FIG. 1 shows Redundancy Analysis of all HITChip datasets collected from Study 1 and Study 2, including in total 95 IBS subjects and 90 healthy controls.

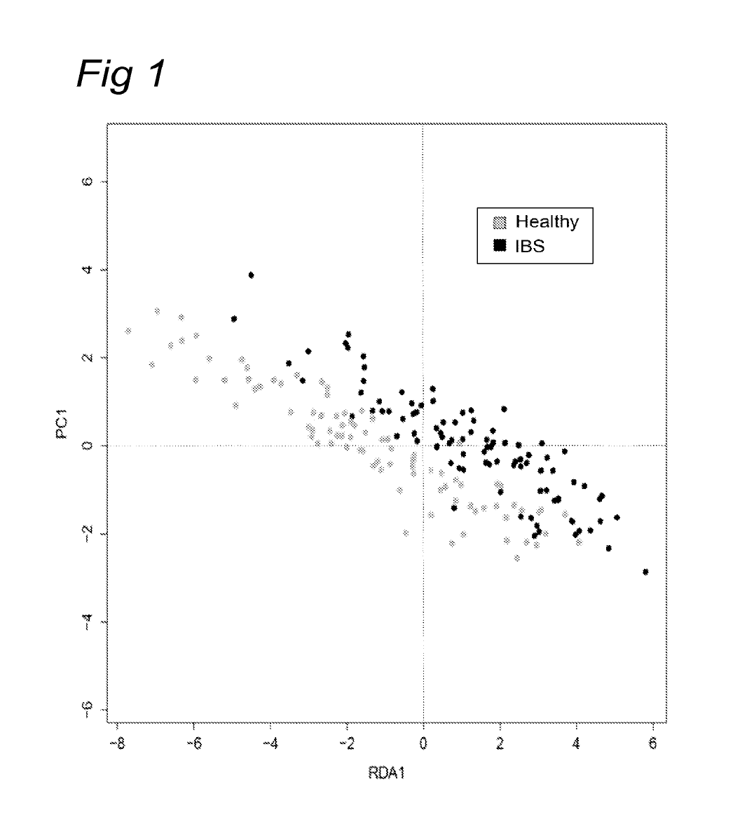

FIG. 2 shows a decision tree for classifying IBS subjects (U) and Healthy controls (H) using hybridization to 4 probes with the indicated Probe ID. Numbers indicate number of subjects in the order H/U reflecting Healthy/IBS.

### SUMMARY OF THE INVENTION

The present invention provides for a method for diagnosing and/or subtyping Irritable Bowel Syndrome (IBS) in a test sample, said method comprising the steps of: a) determining the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Prevotella melaninogenica et rel., Prevotella oralis et rel., Uncultured Bacteroidetes, Tannerella et rel., Parabacteroides distasonis et rel., Allistipes et rel., Bacteroides plebeius et rel., Bacteroides splachnicus et rel., or to the supertaxon Clostridium cluster IV, selected from the taxa Subdoligranulum variabile et rel., Faecalibacterium prausnitzii et rel., Oscillospira guillermondii et rel., Sporobacter termitidis et rel., Ruminococcus callidus et rel., Eubacterium siraeum et rel., Anaerotruncus colihominis et rel., Clostridium cellulosi et rel., Clostridium leptum et rel., Ruminococcus bromii et rel., or to the supertaxon Clostridium cluster IX, said bacteria belonging to the taxon Phascolarctobacterium faecium et rel.; or to the supertaxon Clostridium cluster XVI, said bacteria belonging to the taxon Eubacterium biforme et rel.; or to the supertaxon Clostridium cluster XVII, said bacteria belonging to the taxon Catenibacterium mitsuokai et rel.; or to the supertaxon Proteobacteria, said bacteria belonging to the taxon Xanthomonadaceae; or to the supertaxon Uncultured Clostridiales, selected from the taxa Uncultured Clostridiales I and Uncultured Clostridiales II; or to the supertaxon Uncultured Mollicutes, said bacteria belonging to the taxon Uncultured Mollicutes, and said IBS-increased bacteria being selected from bacteria belonging to the supertaxon Clostridium cluster XIVa, selected from the taxa Dorea formicigenerans et rel., Ruminococcus obeum et rel., Clostridium nexile et rel., Clostridium symbiosum et rel., Outgrouping Clostridium cluster XIVa, Ruminococcus lactaris et rel., Lachnospira pectinoschiza et rel.; in a test sample; b) Comparing said level of said two or more IBS-decreased and/or IBS-increased bacteria in said test sample to a level of said two or more IBS-decreased and/or IBS-increased bacteria in a control sample; and c1) relating a decreased level of said IBS-decreased bacteria and/or an increased level of said IBS-increased bacteria in the test sample compared to the control sample to a diagnosis that the test sample is from a subject suffering from Irritable Bowel Syndrome; and/or c2) relating an increased level of said IBS-increased bacteria or a decreased level of said IBS-decreased bacteria in the test sample compared to the control sample to a diagnosis of whether the test sample is from a subject suffering from IBS-A, IBS-C, or IBS-D.

In an embodiment, step c1) is performed, whereas step c2) is not performed. In another embodiment, step c2) is performed, whereas step c1) is not performed. In yet another embodiment, both steps c1) and c2) are performed.

In an embodiment, said method is for diagnosing IBS, wherein in step a) at least the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Prevotella melaninogenica et rel., Prevotella oralis et rel., Uncultured Bacteroidetes, Tannerella et rel.; or to the supertaxon Clostridium cluster XVII, said bacteria belonging to the taxon Catenibacterium mitsuokai et rel.; or to the supertaxon Proteobacteria, said bacteria belonging to the taxon Xanthomonadaceae; or to the supertaxon Uncultured Clostridiales, said bacteria belonging to the taxon Uncultured Clostridiales I; and said IBS-increased bacteria being selected from bacteria belonging to the supertaxon Clostridium cluster XIVa, selected from the taxa Dorea formicigenerans et rel., Ruminococcus obeum et rel., Clostridium nexile et rel., Clostridium symbiosum et rel., Outgrouping Clostridium cluster XIVa, Ruminococcus lactaris et rel., Lachnospira pectinoschiza et rel.; in a test sample are determined.

In an embodiment, said method is for diagnosing IBS, wherein in step a) the levels of at least one IBS-increased bacteria selected from bacteria belonging to the taxa Dorea formicigenerans et rel., Ruminococcus obeum et rel., and Lachnospira pectinoschiza et rel., and the level of at least one IBS-decreased bacteria selected from bacteria belonging to the taxa Prevotella melaninogenica et rel, Prevotella oralis et rel., and Catenibacterium mitsuokai et rel., are determined.

In an embodiment, said method is for subtyping IBS-A, wherein in step a) the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Uncultured Bacteroidetes, Tannerella et rel., Parabacteroides distasonis et rel., Allistipes et rel., Bacteroides plebeius et rel., Bacteroides splachnicus et rel., or to the supertaxon Clostridium cluster IV, selected from the taxa Subdoligranulum variabile et rel., Faecalibacterium prausnitzii et rel., Oscillospira guillermondii et rel., Sporobacter termitidis et rel., Ruminococcus callidus et rel., Eubacterium siraeum et rel., Anaerotruncus colihominis et rel., Clostridium cellulosi et rel., Clostridium leptum et rel., Ruminococcus bromii et rel., or to the supertaxon Clostridium cluster IX, said bacteria belonging to the taxon Phascolarctobacterium faecium et rel.; or to the supertaxon Clostridium cluster XVI, said bacteria belonging to the taxon Eubacterium biforme et rel.; or to the supertaxon Uncultured Clostridiales, selected from the taxa Uncultured Clostridiales I and Uncultured Clostridiales II; or to the supertaxon Uncultured Mollicutes, said bacteria belonging to the taxon Uncultured Mollicutes, and said IBS-increased bacteria being selected from bacteria belonging to the supertaxon Clostridium cluster XIVa, selected from the taxa Dorea formicigenerans et rel., Ruminococcus obeum et rel., Outgrouping Clostridium cluster XIVa, in a test sample are determined.

In a further embodiment, said method is for subtyping IBS-C, wherein in step a) at least the levels of two or more bacteria belonging to the taxa Prevotella oralis et rel., Bacteroides plebeius et rel., Clostridium stercorarium et rel., Dorea formicigenerans et rel., Clostridium nexile et rel., Catenibacterium mitsuokai et rel., or Xanthomonadaceae in a test sample are determined.

In another embodiment, said method is for subtyping IBS-D, wherein in step a) at least the levels of two or more bacteria belonging to the taxa Dorea formicigenerans et rel., Ruminococcus obeum et rel., Clostridium nexile et rel., Ruminococcus lactaris et rel., Lachnospira pectinoschiza et rel., Catenibacterium mitsuokai et rel., or the uncultured Clostridiales I in a test sample are determined.

In a preferred embodiment, in step a) of the method of the invention the levels of at least one IBS-increased bacteria and at least one IBS-decreased bacteria in said test sample are determined.

In another preferred embodiment, in step a) of the method of the invention the levels of at least one IBS-increased bacteria selected from bacteria belonging to the taxa Dorea formicigenerans et rel., Ruminococcus obeum et rel., and Lachnospira pectinoschiza et rel., and the level of at least one IBS-decreased bacteria selected from bacteria belonging to the taxa Prevotella melaninogenica et rel, Prevotella oralis et rel., and Catenibacterium mitsuokai et rel., in said test sample are determined.

In yet another preferred embodiment, in step a) at least the levels of bacteria belonging to the taxa Dorea formicigenerans et rel., Ruminococcus obeum et rel., and Lachnospira pectinoschiza et rel., and the level of bacteria belonging to the taxa Prevotella melaninogenica et rel, Prevotella oralis et rel., and Catenibacterium mitsuokai et rel., in said test sample are determined.

The level of said one or more bacteria may be measured by determining the level of nucleic acid sequences, amino acid sequences and/or metabolites specific for said one or more bacteria, preferably the level of nucleic acid sequences specific for said one or more bacteria, e.g. 16S rRNA gene sequences or unique genomic sequences of said one or more bacteria.

In an embodiment, the level of said 16S rRNA gene sequences of said one or more bacteria is measured by determining one or more variable regions of said 16S rRNA gene sequences, e.g., one or more of the variable regions V1 and/or V6 of said 16S rRNA gene sequences.

In a suitable embodiment, the levels of nucleic acid sequences specific for said two or more bacteria are determined using PCR or LCR.

The present invention is also directed to a method for diagnosing and/or subtyping Irritable Bowel Syndrome (IBS) in a test sample, said method comprising the steps of: i) providing a test sample; ii) determining the level of at least three nucleic acids capable of hybridising to at least three nucleic acid sequences selected from the nucleic acid sequences of SEQ ID Nos:1-100, or derivatives or fragments thereof deviating by at most 2 nucleotides, and complements, reverse, and reverse complements thereof, under stringent hybridization conditions, in said test sample; ii) comparing the level of said at least three nucleic acids from said test sample to the level of said at least three nucleic acids from a control sample; and iiia) relating the level of said at least three nucleic acids from said test sample to a diagnosis of whether the test sample is from a subject suffering from Irritable Bowel Syndrome; and/or iiib) relating the level of said at least three nucleic acids from said test sample to a diagnosis of whether the test sample is from a subject suffering from IBS-A, IBS-C, or IBS-D.

In a further aspect, the present invention pertains to a method for diagnosing and/or subtyping Irritable Bowel Syndrome (IBS) in a test sample, said method comprising the steps of: i) providing a test sample; ii) determining the level of at least three nucleic acids capable of hybridising to 16S rRNA nucleic acid sequences hybridizing to the complementary strand of any of the nucleic acid sequences SEQ ID NO.:1-100 or fragments of said 16S rRNA nucleic acid sequences hybridizing to the complementary strand of any of the nucleic acid sequences SEQ ID NO.:1-100, and complements, reverse, and reverse complements thereof, under stringent hybridization conditions, in said test sample; ii) comparing the level of said at least three nucleic acids from said test sample to the level of said at least three nucleic acids from a control sample; and iiia) relating the level of said at least three nucleic acids from said test sample to a diagnosis of whether the test sample is from a subject suffering from Irritable Bowel Syndrome; and/or iiib) relating the level of said at least three nucleic acids from said test sample to a diagnosis of whether the test sample is from a subject suffering from IBS-A, IBS-C, or IBS-D.

In an embodiment, in step iiia) an increased level of nucleic acids from said test sample, said nucleic acids being capable of hybridising to nucleic acid sequences selected from the nucleic acid sequences of SEQ ID Nos:1-27, 70-71, 73-77, 99-100, or derivatives or fragments thereof deviating by at most 2 nucleotides, and complements, reverse, and reverse complements thereof, under stringent hybridization conditions, compared to the level of said nucleic acids from said control sample relates to the diagnosis that the subject is suffering from IBS.

In another embodiment, in step iiia) a decreased level of nucleic acids from said test sample, said nucleic acids being capable of hybridising to nucleic acid sequences selected from the nucleic acid sequences of SEQ ID Nos:28-69, 72, 78-98, or derivatives or fragments thereof deviating by at most 2 nucleotides, and complements, reverse, and reverse complements thereof, under stringent hybridization conditions, compared to the level of said nucleic acids from said control sample relates to the diagnosis that the subject is suffering from IBS.

In an embodiment, the level of at least 6 nucleic acid sequences from said test sample is determined. Significance Analysis of Microarrays (SAM) may be used in comparing the levels of said three or more nucleic acid sequence from said test sample with the levels of said three or more nucleic acid sequence from a control sample. Alternatively, Prediction Analysis of Microarray (PAM) may be used in comparing the levels of said three or more nucleic acid sequence from said test sample with the levels of said three or more nucleic acid sequence from a control sample. In another embodiment, Redundancy Analysis is used in comparing the levels of said three or more nucleic acid sequence from said test sample with the levels of said three or more nucleic acid sequence from a control sample.

In an embodiment, the level is determined using a method selected from: hybridization of the nucleic acids in a sample to the nucleic acid sequences having SEQ ID NO.: 1-100, and complements, reverse, and reverse complements thereof, under stringent hybridization conditions; a Polymerase Chain reaction (PCR) or a Ligase Chain Reaction (LCR).

In another aspect, the present invention relates to an array for diagnosing IBS and/or subtyping IBS-A, IBS-C, or IBS-D, said array comprising at least two nucleic acid sequences specifically hybridize to one or more of SEQ ID NOs: 1-100, or derivatives or fragments thereof deviating by at most 2 nucleotides, and complements, reverse, and reverse complements thereof. Said array may comprise at least two nucleic acid sequences selected from the nucleic acid sequences having SEQ ID Nos:1-100. The at least two nucleic acid sequences may be bound to a solid phase matrix. The array may be a DNA or RNA array, and may be a micro-array.

In a further aspect, the present invention is concerned with use of an array of the present invention for diagnosing IBS and/or subtyping IBS-A, IBS-C, or IBS-D.

### DETAILED DESCRIPTION OF THE INVENTION

In the present invention, in a first study a detailed comparison was made between the microbiota of 62 subjects suffering from IBS (defined according to Rome II or III criteria) and 46 healthy subjects. In a second study, a detailed comparison was made between a further 33 IBS subjects and 43 healthy subjects. It has been demonstrated that based on HITChip profiling of DNA extracted from intestinal samples, a distinction can be made between healthy subjects and subjects suffering from IBS (hereinafter also referred to as “IBS subjects”). Subsequently, a detailed comparison was made between the HITChip data from healthy subjects and subjects suffering from IBS using Redundancy Analysis (RDA). This revealed significant differences between healthy subjects and subjects suffering from IBS. These results with a large group of over 150 human subjects, for the first time provided evidence for the use of microbiota to differentiate between healthy subjects and subjects suffering from IBS. Hence, advanced comparisons were made between the HITChip data of healthy subjects and subjects suffering from IBS resulting in the identification of a series of microbial taxa (phylotype-like and genus-like groups) that can be used to differentiate IBS and healthy subjects. Moreover, detailed analysis of the HIT probes showed that a set of 100 HIT probes of each 16-30 nucleotides were found to be significantly different and hybridized to a higher (27) or lower (40) extent in the IBS subjects than in the healthy subjects.

Thus, the present invention relates to a method for diagnosing and/or subtyping Irritable Bowel Syndrome (IBS) in a test sample, said method comprising the steps of: a) determining the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Prevotella melaninogenica et rel., Prevotella oralis et rel., Uncultured Bacteroidetes, Tannerella et rel., Parabacteroides distasonis et rel., Allistipes et rel., Bacteroides plebeius et rel., Bacteroides splachnicus et rel., or to the supertaxon Clostridium cluster IV, selected from the taxa Subdoligranulum variabile et rel., Faecalibacterium prausnitzii et rel., Oscillospira guillermondii et rel., Sporobacter termitidis et rel., Ruminococcus callidus et rel., Eubacterium siraeum et rel., Anaerotruncus colihominis et rel., Clostridium cellulosi et rel., Clostridium leptum et rel., Ruminococcus bromii et rel., or to the supertaxon Clostridium cluster IX, said bacteria belonging to the taxon Phascolarctobacterium faecium et rel.; or to the supertaxon Clostridium cluster XVI, said bacteria belonging to the taxon Eubacterium biforme et rel.; or to the supertaxon Clostridium cluster XVII, said bacteria belonging to the taxon Catenibacterium mitsuokai et rel.; or to the supertaxon Proteobacteria, said bacteria belonging to the taxon Xanthomonadaceae; or to the supertaxon Uncultured Clostridiales, selected from the taxa Uncultured Clostridiales I and Uncultured Clostridiales II; or to the supertaxon Uncultured Mollicutes, said bacteria belonging to the taxon Uncultured Mollicutes, and said IBS-increased bacteria being selected from bacteria belonging to the supertaxon Clostridium cluster XIVa, selected from the taxa Dorea formicigenerans et rel., Ruminococcus obeum et rel., Clostridium nexile et rel., Clostridium symbiosum et rel., Outgrouping Clostridium cluster XIVa, Ruminococcus lactaris et rel., Lachnospira pectinoschiza et rel.; in a test sample; b) Comparing said level of said two or more IBS-decreased and/or IBS-increased bacteria in said test sample to a level of said two or more IBS-decreased and/or IBS-increased bacteria in a control sample; and c1) relating a decreased level of said IBS-decreased bacteria and/or an increased level of said IBS-increased bacteria in the test sample compared to the control sample to a diagnosis that the test sample is from a subject suffering from Irritable Bowel Syndrome; and/or c2) relating an increased level of said IBS-increased bacteria or a decreased level of said IBS-decreased bacteria in the test sample compared to the control sample to a diagnosis of whether the test sample is from a subject suffering from IBS-A, IBS-C, or IBS-D.

As used herein, the term “IBS-increased bacteria” refers to bacteria that are statistically significantly present more abundantly in IBS subjects compared to healthy subjects. The term “IBS-decreased bacteria” as used herein refers to bacteria that are statistically significantly present more abundantly in healthy subjects compared to IBS subjects. IBS-increased bacteria as used herein encompass, without limitation, bacteria belonging to the supertaxon Clostridium cluster XIVa, selected from the taxa Dorea formicigenerans et rel., Ruminococcus obeum et rel., Clostridium nexile et rel., Clostridium symbiosum et rel., Outgrouping Clostridium cluster XIVa, Ruminococcus lactaris et rel., Lachnospira pectinoschiza et rel., Ruminococcus gnavus et rel. IBS-decreased bacteria as used herein encompass, without limitation, bacteria belonging to the supertaxon Bacteroidetes, selected from the taxa Prevotella melaninogenica et rel., Prevotella oralis et rel., Uncultured Bacteroidetes, Tannerella et rel., Parabacteroides distasonis et rel., Allistipes et rel., Bacteroides plebeius et rel., Bacteroides splachnicus et rel., Bacteroides uniformis et rel., Clostridium stercorarium et rel., or to the supertaxon Clostridium cluster IV, selected from the taxa Subdoligranulum variabile et rel., Faecalibacterium prausnitzii et rel., Oscillospira guillermondii et rel., Sporobacter termitidis et rel., Ruminococcus callidus et rel., Eubacterium siraeum et rel., Anaerotruncus colihominis et rel., Clostridium cellulosi et rel., Clostridium leptum et rel., Ruminococcus bromii et rel., or to the supertaxon Clostridium cluster IX, said bacteria belonging to the taxon Phascolarctobacterium faecium et rel.; or to the supertaxon Clostridium cluster XVI, said bacteria belonging to the taxon Eubacterium biforme et rel.; or to the supertaxon Clostridium cluster XVII, said bacteria belonging to the taxon Catenibacterium mitsuokai et rel.; or to the supertaxon Proteobacteria, said bacteria belonging to the taxon Xanthomonadaceae; or to the supertaxon Uncultured Clostridiales, selected from the taxa Uncultured Clostridiales I and Uncultured Clostridiales II; or to the supertaxon Uncultured Mollicutes, said bacteria belonging to the taxon Uncultured Mollicutes

It has been shown in the present study that the levels of these bacteria in an intestinal sample from IBS subjects differ significantly from levels of these bacteria in an intestinal sample from healthy individuals (Table 1 below shows the ratio of the level of the bacteria in healthy subjects over IBS subjects; the grey background indicates bacteria for which the levels are statistically significantly different between IBS subjects and healthy subjects (p<0.05)).

In an embodiment, the level of one or more bacteria belonging to the taxa Ruminococcus gnavus et rel., Bacteroides uniformis et rel., and Clostridium stercorarium et rel. are further determined.

In step a), the level of one or more bacteria belonging to the taxa Ruminococcus gnavus et rel., Dorea formicigenerans et rel., Ruminococcus obeum et rel., Clostridium nexile et rel., Clostridium symbiosum et rel., Outgrouping Clostridium cluster XIVa, Prevotella oralis et rel., Prevotella melaninogenica et rel., Uncultured Bacteroidetes, Parabacteroides distasonis et rel., Allistipes et rel. Subdoligranulum variabile et rel., Faecalibacterium prauznitzii et rel., Sporobacter termitidis et rel., Ruminococcus callidus et rel., Eubacterium biforme et rel., Eubacterium sireaum et rel., Oscillospira guillermondii et rel., the uncultured Clostridiales I and II, Tannerella et rel., Bacteroides plebeius et rel., Bacteroides splachnicus et rel., Bacteroides uniformis et rel., Clostridium stercorarium et rel., Anaerotruncus colihominis et rel., Clostridium cellulosi et rel., Clostridium leptum et rel., Ruminococcus bromii et rel., Phascolarctobacterium faecium et rel., Ruminococcus lactaris et rel., Lachnospira pectinoschiza et rel., Catenibacterium mitsuokai et rel., Xanthomonadaceae, or Uncultured Mollicutes in a test sample is determined.

The term “test sample” as used herein refers to an intestinal sample. Intestinal samples refer to all samples that originate from the intestinal tract, including, without limitation, feces samples, rectal swap samples, but also samples obtained from other sites in the intestinal tract, such as mucosal biopsies, as was shown previously (Zoetendal et al 2002. Appl. Environ. Microbiol. 68:3401-7 and Kerkhoffs et al., 2009, supra). A test sample may be obtained from an IBS subject, from a healthy individual, from a subject with unknown diagnosis of IBS, or from a person with complaints related to the gastro-intenstinal tract. In case of subtyping of IBS, a test sample may be obtained from a subject known to suffer from IBS, or may be from a a subject with unknown diagnosis of IBS. The test sample may have been processed; for example, DNA and/or RNA may have been isolated from feces samples, rectal swap samples, or samples obtained from other sites in the intestinal tract. Preferably, mRNA is isolated from feces samples, rectal swap samples, or samples obtained from other sites in the intestinal tract to provide a test sample comprising mRNA.

The level of said one or more bacteria may be determined using any method known in the art. Such method includes, without limitation, hybridization, and amplification reactions such as polymerase chain reaction (PCR) and ligase chain reaction (LCR).

For clinical diagnostics the use of nucleic acid arrays is highly advantageous as it couples accuracy and speed to quantitative analysis. Nucleic acid arrays are ordered sequences of DNA or RNA that can be used to selectively isolate and later on quantify specific nucleic acid sequences in complex mixtures—by changing the hybridization and washing conditions the specificity of the detected nucleic acid duplexes can be modulated.

The oligonucleotide sequences used to detect a target sequence, whether on nucleic acid arrays or in solution, will be referred to hereinbelow as a “probe”.

Suitable hybridisation conditions (i.e. buffers used, salt strength, temperature, duration) can be selected by the skilled person, on the basis of experience or optionally after some preliminary experiments. These conditions may vary, depending on factors such the size of the probes, the G+C-content of the probes and whether the probes are bound to an array as described below.

Suitable hybridisation conditions are for instance described in Sambrook et al., Molecular Cloning: A Laboratory manual, (1989) 2nd. Ed. Cold Spring Harbour, N.Y.; Berger and Kimmel, “Guide to Molecular Cloning Techniques”, Methods in Enzymology”, (1987), Volume 152, Academic Press Inc., San Diego, Calif.; Young and Davis (1983) Proc. Natl. Acad. Sci. (USA) 80: 1194; Laboratory Techniques in Biochemistry and Molecular Biology, Vol. 24, Hybridization with Nucleic Acid Probes, P. Thijssen, ed., Elsevier, N.Y. (1993).

The hybridisation conditions are preferably chosen such that each probe will only form a hybrid (duplex) with a target sequence with which the probe is essentially complementary, if such a target sequence is present, and otherwise will not form any hybrid. The term “essentially complementary” as used herein does not mean that the complementarity of a probe to a target sequence such as the 16S rRNA gene should be perfect, and mismatches up to 2 nucleotides can be envisaged.

Each probe should at least in part be complementary to a specific target sequence. The probe may be any nucleic acid (i.e. DNA or RNA) but is preferably DNA. The probe will generally have a size of about 10 to 100 base pairs, preferably about 10 to 40 base pairs. The probes may all be of the same size, or may be of different sizes. The probes can be obtained in any suitable manner. For example, knowing the 16S RNA gene sequences of the bacteria identified herein, probes may be synthesized that are complementary to any part of the sequence of such 16S RNA gene sequence, i.e. using an automated DNA-synthesizer or in any other manner known per se. Also, solid phase nucleic acid synthesis techniques may be used, which may result directly in an array with the desired probes. Furthermore, the probes may be obtained using techniques of genetic engineering, for instance by primer extension using the target sequence as a template, and/or by using one or more restriction enzymes, optionally using amplification.

Also, the probes may comprise one or more “alternative nucleosides”. Examples thereof include the bases Inosine (I) and Uracil (U), as well as dUTP and dITP, and these are included within the term “labeled nucleotide analog”. It is to be understood that the presence of such alternative nucleosides does not prevent the probe and its target sequence to be essentially complementary to one another as defined above.

Quantitative nucleic acid-based amplification reactions may also be used to detect and quantify specific nucleic acid sequences in complex mixtures as in the present invention. These include the well known Polymerase Chain Reaction (PCR) and Ligase Chain Reaction (LCR) and modifications thereof (see McPherson & Moller, 2006. PCR, second edition. Taylor & Francis Group; Wiedman et al., 1994. PCR Meth Appl; 3:S51-S64). LCR is a method of DNA amplification similar to PCR but differs from PCR because it amplifies the probe molecule rather than producing amplicons through polymerization of nucleotides. Two probes are used per each DNA strand and are ligated together to form a single polynucleotide. LCR uses both a DNA polymerase enzyme and a DNA ligase enzyme to drive the reaction. In a specific application of LCR, the resulting polynucleotide can be amplified by PCR and analysed separately or, notably when in multiplex samples, hybridized to arrays.

The target for DNA arrays and quantitative nucleic acid-based amplification reactions such as PCR or LCR are nucleic acids, so DNA or RNA. Such nucleic acids include, without limitation, the 16S RNA gene as well as the 16S rRNA itself, directly or after conversion into DNA via the reverse transcriptase reaction. However, also other nucleic acid sequences can be used provided they are sufficiently different and diagnostic between IBS subjects and healthy individuals. These may include DNA sequences, both coding and non-coding, in the genomes of specific microbes that differ in prevalence between healthy and IBS subjects. Comparative genome or transciptome analysis may be a useful tool to identify such DNA sequences.

In the invention described here specific nucleic acid sequences are identified in intestinal microbiota that can be used to discriminate IBS subjects from healthy individuals, allowing IBS subjects to be diagnosed. Numerous nucleic acid isolation methods are available that differ in their approach that includes mechanical or enzymatic lysis and specific purification methods. While all these methods are applicable to intestinal samples, the repeated bead beating method as described by Yu & Morrison (2004. BioTechniques 36:808-812) is among the most efficient ones while enzymatic methods such as those described recently by Ahroos & Tynkynnen (2009. J. Appl. Microbiol. 106:506-514) can be used in combination with automated methods. All methods introduce specific biases but for comparative purposes all methods can be used if used consistently. The obtained nucleic acids may be used as template for PCR or LCR and/or hybridization reactions described above, e.g. using nucleic acid arrays.

The addition “et rel.” behind the genus-like group name (level 2 group name) stands for et relatives, indicating all relatives of this phylogenetic group, i.e., those indicated in Table 3, in the column headed “level 3”. This information, including the indicated 16S rRNA gene sequences, can be used to develop specific PCR primers or LCR probes to detect the one or more members of these groups. In some literature the addition “et rel.” is replaced by “-like” to indicate the fact that the group includes more than one related species. However, this is a rather ambiguous designation and hence all terms with “et rel.” are cleary defined in Table 3, which has been published by Rajilic-Stojaniovic et al. 2009 vide supra. Moreover, the sequences of the probes provided in Tables 2 and 4 can also be used to identify in the 16S rRNA databases all complete or partial 16S rRNA gene sequences that give a match, either completely or even partially. In this way a catalogue of 16S rRNA gene sequences can be obtained that can be used as targets for the development of specific PCR primers or LCR probes to detect these.

In step b) of the method of the present invention, the level of said one or more bacteria in said test sample is compared to a level of said one or more bacteria in a control sample. The control sample may advantageously be derived from a healthy subject, and is preferably treated in the same way as is the test sample. Thus, preferably the control sample is sampled in the same way as is the test sample, if applicable, nucleic acid is isolated in the same way as is the test sample, and, if applicable, hybridization or quantitative amplification is performed under the same conditions to allow a fair comparison of the test sample and control sample. It is not necessary to determine the level of said one or more bacteria in a control sample each time a test sample is measured; once the level of said one or more bacteria is reliably determined in a control sample, the level values may be stored, e.g., in a computer, and used for the comparative purposes herein set forth.

The level of said one or more bacteria in a test sample is compared to the same bacteria in a control sample, for example, the level of Ruminococcus obeum et rel. in a test sample is compared to the level of Ruminococcus obeum et rel. in a control sample, the level of Bacteroides splachnicus et rel. in a test sample is compared to the level of Bacteroides splachnicus et rel. in a control sample, and the like.

In step c1) of the method of the present invention, an increased level of IBS-increased bacteria and/or a decreased level of IBS-decreased bacteria is related to a diagnosis that the test sample is from a subject suffering from Irritable Bowel Syndrome.

In step c2) of the method of the present invention, an increased level of IBS-increased bacteria and/or a decreased level of IBS-decreased bacteria is related to a diagnosis of whether the test sample is from a subject suffering from IBS-A, IBS-C, or IBS-D.

As used herein, the level of one or more bacteria in a test sample is increased when it is significantly higher than the level of said one or more bacteria in a control sample. It is also considered increased when the level of one or more bacteria in the test sample is at least 5%, such as 10%, 15%, 20%, 25%, 30%, 35%, 40%, 45%, 50% higher than the corresponding one or more bacteria in the control sample.

As used herein, the level of one or more bacteria in a test sample is decreased when it is significantly lower than the level of said one or more bacteria in a control sample. It is also considered decreased when the level of one or more bacteria in the test sample is at least 5%, such as 10%, 15%, 20%, 25%, 30%, 35%, 40%, 45%, 50% lower than the corresponding one or more bacteria in the control sample.

In an embodiment, step c1) is performed, whereas step c2) is not performed. In another embodiment, step c2) is performed, whereas step c1) is not performed. In yet another embodiment, both steps c1) and c2) are performed. For test samples of unknown origin, i.e. of which it is not known whether it is from an IBS subject or from a healthy individual, steps a), b) and c1) may be performed to diagnose IBS. In such case, it may be advantageous to perform both steps c1) and c2) to simultaneously diagnose and subtype IBS. For test samples obtained from an IBS subject, it may be sufficient to perform steps a), b), and c2) in order to subtype the IBS.

In an embodiment, said method is for diagnosing IBS, wherein in step a) at least the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Prevotella melaninogenica et rel., Prevotella oralis et rel., Uncultured Bacteroidetes, Tannerella et rel.; or to the supertaxon Clostridium cluster XVII, said bacteria belonging to the taxon Catenibacterium mitsuokai et rel.; or to the supertaxon Proteobacteria, said bacteria belonging to the taxon Xanthomonadaceae; or to the supertaxon Uncultured Clostridiales, said bacteria belonging to the taxon Uncultured Clostridiales I; and said IBS-increased bacteria being selected from bacteria belonging to the supertaxon Clostridium cluster XIVa, selected from the taxa Dorea formicigenerans et rel., Ruminococcus obeum et rel., Clostridium nexile et rel., Clostridium symbiosum et rel., Outgrouping Clostridium cluster XIVa, Ruminococcus lactaris et rel., Lachnospira pectinoschiza et rel.; in a test sample are determined.

In an embodiment, said method is for diagnosing IBS, wherein in step a) the levels of at least one IBS-increased bacteria selected from bacteria belonging to the taxa Dorea formicigenerans et rel., Ruminococcus obeum et rel., and Lachnospira pectinoschiza et rel., and the level of at least one IBS-decreased bacteria selected from bacteria belonging to the taxa Prevotella melaninogenica et rel, Prevotella oralis et rel., and Catenibacterium mitsuokai et rel., are determined.

In an embodiment, said method is for subtyping IBS-A, wherein in step a) the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Uncultured Bacteroidetes, Tannerella et rel., Parabacteroides distasonis et rel., Allistipes et rel., Bacteroides plebeius et rel., Bacteroides splachnicus et rel., or to the supertaxon Clostridium cluster IV, selected from the taxa Subdoligranulum variabile et rel., Faecalibacterium prausnitzii et rel., Oscillospira guillermondii et rel., Sporobacter termitidis et rel., Ruminococcus callidus et rel., Eubacterium siraeum et rel., Anaerotruncus colihominis et rel., Clostridium cellulosi et rel., Clostridium leptum et rel., Ruminococcus bromii et rel., or to the supertaxon Clostridium cluster IX, said bacteria belonging to the taxon Phascolarctobacterium faecium et rel.; or to the supertaxon Clostridium cluster XVI, said bacteria belonging to the taxon Eubacterium biforme et rel.; or to the supertaxon Uncultured Clostridiales, selected from the taxa Uncultured Clostridiales I and Uncultured Clostridiales II; or to the supertaxon Uncultured Mollicutes, said bacteria belonging to the taxon Uncultured Mollicutes, and said IBS-increased bacteria being selected from bacteria belonging to the supertaxon Clostridium cluster XIVa, selected from the taxa Dorea formicigenerans et rel., Ruminococcus obeum et rel., Outgrouping Clostridium cluster XIVa, in a test sample are determined.

In another embodiment, said method is for subtyping IBS-A, wherein in step a) the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Parabacteroides distasonis et rel., Allistipes et rel., Bacteroides splachnicus et rel., or to the supertaxon Clostridium cluster IV, selected from the taxa Subdoligranulum variabile et rel., Faecalibacterium prausnitzii et rel., Oscillospira guillermondii et rel., Sporobacter termitidis et rel., Ruminococcus callidus et rel., Eubacterium siraeum et rel., Anaerotruncus colihominis et rel., Clostridium cellulosi et rel., Clostridium leptum et rel., Ruminococcus bromii et rel., or to the supertaxon Clostridium cluster IX, said bacteria belonging to the taxon Phascolarctobacterium faecium et rel.; or to the supertaxon Clostridium cluster XVI, said bacteria belonging to the taxon Eubacterium biforme et rel.; or to the supertaxon Uncultured Clostridiales, selected from the taxa Uncultured Clostridiales I and Uncultured Clostridiales Ilin a test sample are determined.

The bacteria belonging to these taxa are unique for IBS-A subtyping.

In a further embodiment, said method is for subtyping IBS-C, wherein in step a) at least the levels of two or more bacteria belonging to the taxa Prevotella oralis et rel., Bacteroides plebeius et rel., Dorea formicigenerans et rel., Clostridium nexile et rel., Catenibacterium mitsuokai et rel., or Xanthomonadaceae in a test sample are determined.

In another embodiment, said method is for subtyping IBS-D, wherein in step a) at least the levels of two or more bacteria belonging to the taxa Dorea formicigenerans et rel., Ruminococcus obeum et rel., Clostridium nexile et rel., Ruminococcus lactaris et rel., Lachnospira pectinoschiza et rel., Catenibacterium mitsuokai et rel., or the uncultured Clostridiales I in a test sample are determined.

It is preferred that the levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more bacteria which are present in statistically significantly different levels between IBS subjects and healthy subjects, said bacteria being selected from IBS-decreased bacteria and IBS-increased bacteria, said IBS-decreased bacteria as defined hereinabove are determined to allow an even more reliable diagnosis of IBS and/or subtyping of IBS-A, IBS-C and/or IBS-D. Furthermore, any other statistical operation to the levels of said microbial groups available to persons skilled in the art also may allow for a more reliable diagnosis of IBS.

The level of said one or more bacteria may be measured by determining the levels of nucleic acid sequences, amino acid sequence and/or metabolites specific for said one or more bacteria, preferably the level of nucleic acid sequences specific for said one or more bacteria.

One of the most researched microbial nucleic acids is that of the 16S rRNA. This 16S rRNA, also known as small subunit (SSU) RNA, is encoded by an approximately 1500 by gene that is present in a variable number of copies, usually 1-10 per microbial genome. The nucleotide sequence of the 16S rRNA genes is frequently used in diagnostics as it shows differences between microbial species. In fact 16S rRNA gene sequences are instrumental in defining the taxonomic position of microbes. Moreover, these 16S rRNA sequences may also identify microbes that have not yet been cultured but are only known because of the presence of a 16S rRNA gene sequence. In case this gene sequence differs significantly (usually less than 98% similarity) from the 16S rRNA gene sequence of a known species, this is indicated as a new phylotype (a microbe that has not been cultured yet). However, a growing number of microbes are brought into culture and otherwise described by sequence analysis of their complete or partial genomes. Up to now over several thousands of microbial genomes have been sequenced and are publicly available (see http://genomesonline.org or http://www.ncbi.nlm.nih.gov). Many more are to follow either after their isolation or from metagenome projects that aim to sequence the entire microbial DNA present in an ecosystem, such as Human Microbiome Project aiming to determine the metagenome of the human microbiota (see http://nihroadmap.nih.gov/hmp/).

A growing database of over a million microbial 16S rRNA sequences can be found in publicly available databases such as http://www.arb-silva.de (Pruesse et al., 2007. Nucleic Acid Res. 35:7188) and http://rdp.cmu.mse.edu (Cole et al., 2008. Nucleic Acids Res. 35 (Database issue): D169-D172). It has been well-established that the 16S rRNA sequence contains a limited number of variable regions of several dozens of nucleotides, termed V1-V8, that are targets for developing nucleic acid probes, PCR primers or LCR probes. By analyzing the variable regions in the microbes that are found in the human intestinal tract, it was observed that the most diagnostic information for developing nucleic acid probes were the V1 and V6 regions (Rajilic-Stojanovic et al., 2009, supra). Hence, based on the sequences of these variable regions a total of over 3,699 unique oligonucleotide probes of around 16-30 nucleotides have been developed that are present on the so called Human Intestinal Tract (HIT) Chip, a phylogenetic microarray (Rajilic-Stojanovic et al 2009, supra). These oligonucleotides are called HIT probes. Hybridization to the HIT probes can be used to deduce what microbe is present and allows its taxonomic identification at different level, the most important ones including genus-like groups (sequence similarity>90%—so called level 2 groups) and phylotype-like groups (sequence similarity>98%—so called level 3 groups) (Rajilic-Stojanovic et al 2009, supra). Table 3 defines the identified groupings even when the systematic names of the involved bacterial species is changing due to advanced taxonomic insight.

“Percentages (%) sequence identity” refers to the percentage identical nucleotides between two sequences and can be determined using for example pairwise local alignment tools such as the program “water” of EmbossWlN (version 2.10.0) using default parameters, (gap opening penalty 10.0 and gap extension penalty 0.5, using Blosum62 for proteins and DNAFULL matrices for nucleic acids) or “Bestfit” of GCG Wisconsin Package, available from Accelrys Inc., 9685 Scranton Road, San Diego, Calif. 92121-3752 USA, using default parameters. Alternatively, BLAST analysis using default settings may also be used, such as nucleotide Blast of NCIMB, with a gap creation penalty 11 and gap extension penalty 1.

Thus, the level of said one or more bacteria is preferably measured by determining the level of specific nucleic acid sequences in said test sample, which nucleic acid sequences are preferably 16S rRNA gene sequences of said one or more bacteria, more preferably one or more variable regions of said 16S rRNA gene sequences, e.g., one or more of the variable regions V1 and/or V6 of said 16S rRNA gene sequences.

The disclosed microbial groups as well as the differentiating oligonucleotide probes can serve alone or in combination as biomarkers for IBS subjects. A biomarker, or biological marker, is in general a substance used as an indicator of a biologic state. Biomarkers can include a variety of stable macromolecular molecules, including nucleic acids, proteins or lipids but also metabolites or a combination thereof. Of particular interest are nucleic acids, including DNA and RNA, that are present in the intestinal microbiota as they are stable but can be isolated easily. However, also proteins encoded by the said DNA can be considered useful biomarkers, notably when they are stable.

Starting from the microbial groups, bacteria and probes described herein, persons skilled in the art can deduce LCR, PCR or hybridization probes to specifically discriminate IBS subjects from healthy subjects using intestinal microbiota as target. In some cases even discriminatory microbial groups are identified that are specifically affected in one or more specific types of IBS. Affected in this context means either more or less prevalent in IBS subjects, allowing for biomarker development for specific IBS-subtypes such as IBS-C, IBS-A and IBS-D.

The identification of the microbial groups that are specifically affected also allows new classification of IBS and its subsequent therapy. This therapy may consist of the consumption of correcting microbes, conforming to the definition of probiotics (see http://www.isapp.net/). In addition, consumption of prebiotics can be envisaged that affect the microbial composition (http://www.isapp.net/). Finally, pharmaceutical preparations can be envisaged that affect the microbiota in such a way that the identified defects are corrected. Here ‘defects’ are defined as ‘deviating from healthy subjects with regard to gastro-intestinal microbiota’.

It is evident that the present diagnosis of IBS should be improved and analysis of the gut microbiota is an important diagnostic tool. However, the classification of IBS into the IBS-C, IBS-D and IBS-A types according to the Rome criteria is mainly based on form and frequency of stool samples and hence subjective, undefined and biased (Thompson et al., 1989. Gastroenterol Int 2:92-95; Longstreth et al., 2006, supra; Thompson, 2006. Gastroenterology 130: 1552-1556). The traditional classification of IBS subjects based on the Rome criteria does not provide a solid basis for therapy and this hampers treatment of the IBS subjects.

Based on the microbiota analysis and detection of the identified oligonucleotides specific for IBS (probes having SEQ ID Nos:1-27, 70-71, 73-77, 99-100) and Healthy subjects (probes having SEQ ID Nos:28-69, 72, 78-98) (see Tables 2 and 4) of the invention new, rational and unbiased differentiation of the IBS subjects can be realized. It is envisaged that this results in classifications that are useful in combination with specific treatments and thus improving the efficacy of therapies. As such, the invention will allow for differentiating IBS subjects based upon the microbiota in their GI tract. Hence, the classification of IBS following microbiota analysis is a preferred embodiment of the invention. Inspection of the major differences in microbial composition in the IBS-C, IBS-D and IBS-A allows the definition of IBS subtypes based on specific microbial composition.

Starting from the present invention, it may be possible to determine the level of the bacterial taxa as described hereinabove. However, an alternative way of diagnosing and/or subtyping IBS is to use the selective hybridization probes of SEQ ID NO.:1-100 identified herein, or complements, reverse, or reverse-complements thereof. The hybridization probes of SEQ ID NO.:1-100 may be used as such for hybridization with nucleic acids isolated from a test sample to provide a diagnosis of IBS and/or to subtype IBS. Alternatively, probes with up to 2 nucleotide mismatches in comparison to SEQ ID NO.:1-100, or complements, reverse, or reverse-complements thereof, may be used. Alternatively, the probes may be used to identify 16S rRNA nucleic acid sequences useful for diagnosing IBS and/or subtyping IBS. To this end, the nucleic acid sequences of SEQ ID NO.:1-100, or derivatives or fragments thereof deviating by at most 2 nucleotides, or complements, reverse, or reverse-complements thereof, may be used to perform a search in well-known public nucleic acid sequence databases in order to identify those 16S rRNA sequences that are useful in diagnosing IBS and/or subtyping IBS. In the present case, the SILVA and RDP databases were searched for 16S rRNA gene sequences using the nucleic acid sequences of SEQ ID NO.:1-100 allowing up to 2 mismatches from these nucleic acid sequences. This resulted in multiple hits for each of the nucleic acid sequences. It is to be understood that the 16S rRNA sequences thus identified, as well as sequences derived therefrom, may also be used to diagnose IBS and/or subtype IBS. For example, nucleic acid sequences suitable for hybridization reactions (herein also referred to as “probes”) useful to diagnose IBS and/or subtype IBS may be identified starting from the 16S rRNA sequences identified using nucleic acid sequences of SEQ ID NO.:1-100, or derivatives or fragments thereof deviating by at most 2 nucleotides, or complements, reverse, or reverse-complements thereof. Alternatively, the 16S rRNA sequences identified using nucleic acid sequences of SEQ ID NO.:1-100, or derivatives or fragments thereof deviating by at most 2 nucleotides, or complements, reverse, or reverse-complements thereof, may be used to develop amplification primers for use in amplification reactions, e.g., for use in PCR or LCR reactions. Such amplification reactions may also be used to diagnose IBS and/or subtype IBS. Sequences which are the complement, reverse or reverse-complement of the nucleic acid sequences of SEQ ID Nos:1-100, derivatives or fragments thereof deviating by at most 2 nucleotides, 16S rRNA sequences identified using nucleic acid sequences of SEQ ID NO.:1-100, or derivatives or fragments thereof deviating by at most 2 nucleotides, may also be used in the methods of the invention.

The present invention is also directed to a method for diagnosing and/or subtyping Irritable Bowel Syndrome (IBS) in a test sample, said method comprising the steps of: i) providing a test sample; ii) determining the level of at least three nucleic acids capable of hybridising to at least three nucleic acid sequences selected from the nucleic acid sequences of SEQ ID Nos:1-100, or derivatives or fragments thereof deviating by at most 2 nucleotides, and complements, reverse, and reverse complements thereof, under stringent hybridization conditions, in said test sample; ii) comparing the level of said at least three nucleic acids from said test sample to the level of said at least three nucleic acids from a control sample; and iiia) relating the level of said at least three nucleic acids from said test sample to a diagnosis of whether the test sample is from a subject suffering from Irritable Bowel Syndrome; and/or iiib) relating the level of said at least three nucleic acids from said test sample to a diagnosis of whether the test sample is from a subject suffering from IBS-A, IBS-C, or IBS-D.

In an alternative method of the invention, in step i) the level of at least three nucleic acids capable of hybridising to 16S rRNA nucleic acid sequences hybridizing to the complementary strand of any of the nucleic acid sequences SEQ ID NO.:1-100 or fragments of said 16S rRNA nucleic acid sequences hybridizing to the complementary strand of any of the nucleic acid sequences SEQ ID NO.:1-100, and complements, reverse, and reverse complements thereof, under stringent hybridization conditions, in said test sample, is determined.

The term “level” as used in combination with nucleic acids or nucleic acid sequences may refer to expression level as determined using mRNA, or the amount of genomic DNA present in a sample.

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

In an embodiment, step iiia) is performed, whereas step iiib) is not performed. In another embodiment, step iiib) is performed, whereas step iiia) is not performed. In yet another embodiment, both steps iiia) and iiib) are performed. For test samples of unknown origin, i.e. of which it is not known whether it is from an IBS subject or from a healthy individual, steps i), ii) and iiia) may be performed to diagnose IBS. In such case, it may be advantageous to perform both steps iiia) and iiib) to simultaneously diagnose and subtype IBS. For test samples obtained from an IBS subject, it may be sufficient to perform steps i), ii), and iiib) in order to subtype the IBS.

In an embodiment, in step iiia) an increased level of nucleic acids from said test sample, said nucleic acids being capable of hybridising to nucleic acid sequences selected from the nucleic acid sequences of SEQ ID Nos:1-27, 70-71, 73-77, 99-100, or derivatives or fragments thereof deviating by at most 2 nucleotides, and complements, reverse, and reverse complements thereof, under stringent hybridization conditions, compared to the level of said nucleic acids from said control sample relates to the diagnosis that the subject is suffering from IBS.

In a further embodiment, in step iiia) a decreased level of nucleic acids from said test sample, said nucleic acids being capable of hybridising to nucleic acid sequences selected from the nucleic acid sequences of SEQ ID Nos:28-69, 72, 78-98, or derivatives or fragments thereof deviating by at most 2 nucleotides, and complements, reverse, and reverse complements thereof, under stringent hybridization conditions, compared to the level of said nucleic acids from said control sample relates to the diagnosis that the subject is suffering from IBS.

As such, the nucleic acid or nucleotide sequences of SEQ ID NO.:1-100, or derivatives or fragments thereof deviating from SEQ ID NO.:1-100 by at most 2 nucleotides, or the complement, reverse, or reverse-complement thereof, may be used to discriminate between healthy subjects and subjects suffering from IBS, as well as between subject suffering from the various subtypes of IBS: IBS-A, IBS-C and IBS-D. Although two nucleic acid sequences selected from the group consisting of SEQ ID NO.:1-100 may suffice for diagnosing IBS and/or subtyping IBS-A, IBS-C and/or IBS-D, it is preferred that at least 3, 4, 5, 6, 7, 8, 9, 10, 12, 15, 17, 20, 25, 30, 35, 40, or more nucleic acid sequences selected from the group consisting of SEQ ID Nos.:1-100 are employed in the method of the present invention. In an embodiment, all nucleic acid sequences of SEQ ID NO.:1-100, or derivatives or fragments thereof deviating by at most 2 nucleotides, or the complement, reverse, or reverse-complement thereof, are employed for diagnosing and/or subtyping IBS in a test sample.

The levels of the nucleic acid sequences in a test sample may be subjected to statistical and/or bioinformatical analysis to obtain analyzed data; and the analyzed data of said test sample may be compared to analyzed data from a control sample, to provide a diagnosis of whether the test sample is from a subject suffering from Irritable Bowel Syndrome. For example, hybridization patterns on a micro-array comprising the nucleic acid sequences having SEQ ID NO: 1-100. In this method, the hybridization data generated using SEQ ID Nos.:1-100 may be processed using statistical and/or bioinformatical analysis such as Principal Component Analysis (PCA) and/or Redundancy Analysis (RDA). The analyzed data may then be compared to analyzed data from a control sample which has been subject to the same statistical and/or bioinformatical analysis, which may relate to a diagnosis of whether the test sample is from a subject suffering from IBS.

In an embodiment, Significance Analysis of Microarrays (SAM) is used in comparing the levels of said three or more nucleic acid sequence from said test sample with the levels of said three or more nucleic acid sequence from a control sample. The person skilled in the art is capable of performing SAM analysis. SAM analysis is described in detail by Tusher et al. (Proc Natl Acad Sci USA, 2001, vol 98:5116-5121), which is herein incorporated by reference.

In another embodiment, Prediction Analysis of Microarray (PAM) is used in comparing the levels of said three or more nucleic acid sequence from said test sample with the levels of said three or more nucleic acid sequence from a control sample. The person skilled in the art is capable of performing PAM analysis. PAM analysis is described in detail by Tibshirani et al. (Proc Natl Acad Sci USA, 2002, vol 99:6567-6572), which is herein incorporated by reference.

In yet another embodiment, Redundancy Analysis (RDA) is used in comparing the levels of said three or more nucleic acid sequence from said test sample with the levels of said three or more nucleic acid sequence from a control sample. The person skilled in the art is capable of performing RDA analysis. RDA analysis is described in detail by Leps and Smilauer (2003. Cambridge University Press: Multivariate analysis of ecological 780 data using CANOCO), which is herein incorporated by reference.

The level may be determined using a method selected from: hybridization of the nucleic acids in a sample to the nucleic acid sequences having SEQ ID NO.:1-100, and complements, reverse, and reverse complements thereof, under stringent hybridization conditions; a Polymerase Chain reaction (PCR) or a Ligase Chain Reaction (LCR).

In yet another aspect, the invention pertains to a method for diagnosing and/or subtyping Irritable Bowel Syndrome (IBS) in a test sample, said method comprising the steps of: i) determining the level of amplification of at least three nucleic acid sequences from a test sample using one or more of the nucleic acid sequences of SEQ ID NO.: 1-100, or derivatives or fragments thereof deviating by at most 2 nucleotides, or nucleic acids capable of hybridising to 16S rRNA nucleic acid sequences hybridizing to the complementary strand of any of the nucleic acid sequences SEQ ID NO.:1-100 or fragments of said 16S rRNA nucleic acid sequences hybridizing to the complementary strand of any of the nucleic acid sequences SEQ ID NO.:1-100, and complements, reverse, and reverse complements thereof; ii) comparing the level of amplification of said at least three nucleic acid sequences from said test sample to the level of amplification of said at least three nucleic acid sequences from a control sample; and iiia) relating the level of amplification of said at least three nucleic acid sequences from said test sample compared to the level of amplification of said at least three nucleic acid sequences from a control sample to a diagnosis of whether the test sample is from a subject suffering from Irritable Bowel Syndrome; and/or iiib) relating the level of amplification of said at least three nucleic acid sequences from said test sample compared to the level of amplification of said at least three nucleic acid sequences from a control sample to a diagnosis of whether the test sample is from a subject suffering from IBS-A, IBS-C, or IBS-D.

It is to be noted that also the levels of one or more bacteria belonging to the taxa Collinsella (see Table 1) may be used for diagnosing and subtyping IBS in the method of the present invention. In particular, they may be used for subtyping IBS-A in the methods of the present invention. A decreased level of two or more bacteria belonging to the taxa Collinsella in the test sample relates to a diagnosis that the test sample is from a subject suffering from IBS-A.

In another aspect, the present invention provides for an array for diagnosing IBS and/or subtyping IBS-A, IBS-C, or IBS-D, said array comprising at least two nucleic acid sequences having the nucleic acid sequence of SEQ ID NOs: 1-100, or derivatives or fragments thereof deviating by at most 2 nucleotides, or complements, reverse, and reverse complements thereof. It was found that the nucleotide sequences mentioned were highly suitable for diagnosing IBS from 3,699 unique nucleotide sequences that were tested.

Preferably, said array comprises at least two nucleic acid sequences selected from the nucleic acid sequences having SEQ ID Nos:1-100. The at least two nucleic acid sequences may be bound to a solid phase matrix. The array may be a DNA or RNA array, and may be a micro-array.

In a final aspect, the present invention is concerned with the use of an array of the invention for diagnosing IBS and/or subtyping IBS-A, IBS-C, or IBS-D.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition of the invention may comprise additional component(s) than the ones specifically identified, said additional component(s) not altering the unique characteristics of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The terms “increased level” and “decreased level” as used throughout this document refers to a significantly increased level or significantly decreased level. Generally, a level in a test sample is increased or decreased when it is at least 5%, such as 10%, 15%, 20%, 25%, 30%, 35%, 40%, 45%, 50% higher or lower, respectively, than the corresponding level in a control sample.

All patent and literature references cited in the present specification are hereby incorporated by reference in their entirety.

It will be clear that the above description and figures is included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

TABLE 1
Significantly different level 2 groups between IBS and healthy subjects. The
ratio of the average hybridization signal of healthy controls and IBS subjects (all
together and grouped according to IBS-c, IBS-d and IBS-a) is presented together with
the significance level (as indicated by a t-test; grey indicates significance at the
p < 0.05 level).

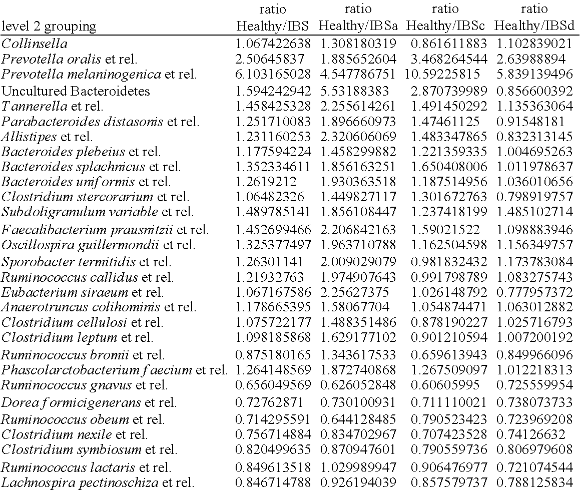

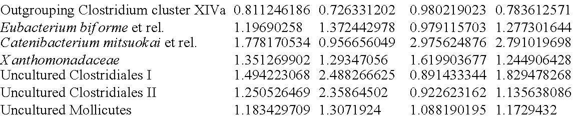

TABLE 2
Identification, sequence and analysis of the
HIT probes that differ significantly at the
p < 0.05 level between IBS subjects and
healthy controls. The oligonucleotides with
SEQ ID NO: 1-27 that showed a significantly
higher hybridization signal in the IBS than
the healthy subjects and the oligonucleotides
with SEQ ID 28-67 that showed the opposite,
are indicated with their nucleotide sequence
(3′ to 5′).

| | SEQ |
|---|---|
| Sequence 5′ to 3′ direction | ID |
| (T = U in RNA) | NO. |
| GCCGCTCAGTCACAATCCTC | 1 |
| GCCACTAGAAATAGATCAAATCCAC | 2 |
| GCCGCTCAGTCACAAAACTCTTCA | 3 |
| CCGAAGTTTCAATAAAGTAATTCCCG | 4 |
| GCCACTAGAATTAAATTAAATCGACCG | 5 |
| CGAAGTCTCAATGAAATATTTCCCG | 6 |
| CACTAGAAATAGATCAAATCCACCG | 7 |
| GCCACTCAGTCACAGTCTCTC | 8 |
| GCCGCTCAGTCACCAAGG | 9 |
| GCCGCTCAGTCACAACACTC | 10 |
| GCCGCTCAGTCACAAAACC | 11 |
| GCCGCTCAGTCACAAACGGA | 12 |
| GCCGCTCAGTCACTGTCC | 13 |
| GCCACTAGAATTAAATTATATCGACCG | 14 |
| GCCACTAGAATTAAATCATATCGACC | 15 |
| TGTCTCCGCTGCCCCGAA | 16 |
| TAAATCATATCGACCGAAGTTTCAATAAAA | 17 |
| AAATTATATCGACCGAAGTTTCAATAAAG | 18 |
| GCCACTAGAAATAAATCAAATCCACC | 19 |
| AGCAAGCTCCTCCTTCAGCG | 20 |
| ATCCTCTTCATCCGAAGAATCTAAG | 21 |
| GCCGCTTTCCACTCTTAACTTCAA | 22 |
| AGAAATCCGTCAAGGTGCTTCGC | 23 |
| GAAGTTTCAATAAAATAATTCCCGTTCG | 24 |
| TGTCCTCTTCCTCCGAAGATTCTG | 25 |
| CCGAAGTTTCAATAAAATAATTCCCG | 26 |
| GATCCGTTTAAGGTGCTTCGTTCG | 27 |
| TGTCTCTGCGTCCCGAAGGAAAA | 28 |
| TGTCTCTGCGTCCCGAAGGAATA | 29 |
| TGTCTCTGCGTCCCGAAGGAAA | 30 |
| GCCACTGTCCTCTGCTTCAC | 31 |
| ATCGTCGCAGGATGTCAAGACTTG | 32 |
| CAAGCTCCTCTCAGCTCCG | 33 |
| GGCTGACATGTCTCCACATCATTC | 34 |
| CGTCGCAGGATGTCAAGACTTG | 35 |
| ACCGTCGCAGGATGTCAAGAC | 36 |
| TGTCTCTGCTGTCCCGAAGGAAA | 37 |
| GCCACTGTCCTCTGCTTCGAA | 38 |
| ATCGTCAAGGGATGTCAAGACTTG | 39 |
| TGCGTCGCAGGATGTCAAGAC | 40 |
| CATTCAGTTGCAATTCAAGCCCGG | 41 |
| GCCACTTTCCTCTACATCCATTG | 42 |
| GGATTTCACACATCTCTGTGCTA | 43 |
| TTCGTCAAGGGATGTCAAGACTTG | 44 |
| GTTCGTCAAGGGATGTCAAGAC | 45 |
| GCCACTCGATTTGAAGAGCAAGC | 46 |
| GCCACTAACCGCTCCAATAGTAAA | 47 |
| GATTTGAAGAGCAAGCTCCTCATC | 48 |
| GCCACTCGATCAAGGAAGCAAG | 49 |
| TTCACAACTGCCTTGCGGCTGA | 50 |
| CCTCTTTCCACAGATTCTCGTTCG | 51 |
| CGATTTGAAGAGCAAGCTCCTCA | 52 |
| GAATCCGTAATCAAGCTTCGTTCG | 53 |
| TTCTCCTGCAATTCAAGCCCGG | 54 |
| TCGTTAGCAGGATGTCAAACCCTG | 55 |
| ATGCACCTGCAATTCAAGCCCG | 56 |
| CAAGCTCCTCATCTCTCGTTCG | 57 |
| TGTCTCCTTGCTCCGAAGAGAAA | 58 |
| TGTCTCCTTGCTCCGAAGAGAAAA | 59 |
| TGTCTCCTTGCTCCGAAGAGATTA | 60 |
| TGTCTCGATGTCCCGAAGGATTTC | 61 |
| AGAGCAAGCTCCTCATCTCTCG | 62 |
| GCCACTAGATTGTAGAAAAAGCAAG | 63 |
| GCACCTAATGCATCTCTGCTTCG | 64 |
| GAAGCAAGCTTCCTCTCTCTCG | 65 |
| CAAGCTCCTCTTGATTCCGTTCG | 66 |
| AGAGAATTATTAGCAAGCTAGCAATTC | 67 |

TABLE 3
Classification of phylotypes identified in the present invention based on the 16S rRNA gene sequence
similarity with accession number of the 16S rRNA gene sequence. Level 1 corresponds to the phylum, or in
case of Firmicutes to the Clostridium cluster; Level 2 includes groups of sequences with 90% or more
sequence similarity; Level 3 represents unique phylotypes that were defined as species for cultivated
microorganisms, or representatives of each monophyletic group with ≧98% sequence identity for clones
corresponding to uncultured microorganisms (herein identified as “relatives” or “et rel.”).

| | | | Accession |
|---|---|---|---|
| Level 1 | Level 2 | Level 3 | number |
| Actinobacteria | Actinomycetaceae | Arcanobacterium pyogenes | M29552 |
| | | Actinomyces naeslundii | M33911 |
| | | Uncultured bacterium clone Eldhufec234 | AY920109 |
| | | Uncultured bacterium clone Eldhufec081 | AY919956 |
| | | uncultured bacterium Z650 | AY979340 |
| | | uncultured bacterium NH01 | AY978941 |
| | Atopobium | Atopobium parvulum | AF292372 |
| | | Atopobium minutum | M59059 |
| | Bifidobacterium | Bifidobacterium breve | AB006658 |
| | | Bifidobacterium thermophilum | AB016246 |
| | | Bifidobacterium angulatum | D86182 |
| | | Bifidobacterium dentium | D86183 |
| | | Bifidobacterium infantis | D86184 |
| | | Bifidobacterium pseudocatenulatum | D86187 |
| | | Bifidobacterium gallicum | D86189 |
| | | Bifidobacterium pseudolongum | D86194 |
| | | Bifidobacterium bifidum | M38018 |
| | | Bifidobacterium adolescentis | M58729 |
| | | Bifidobacterium catenulatum | M58732 |
| | | Bifidobacterium longum | M58739 |
| | | Bifidobacterium sp. CB8 | AB064925 |
| | | Uncultured bacterium clone Eldhufec082 | AY919957 |
| | | uncultured bacterium (human infant) L14E | AF253371 |
| | | uncultured bacterium (human infant) N14A | AF253397 |
| | | uncultured bacterium Adhufec069rbh | AY471706 |
| | | uncultured Bifidobacterium sp. 15D | AF275886 |
| | | uncultured Bifidobacterium sp. 13D | AF275884 |
| | | Bifidobacterium sp. PL1 | AF306789 |
| | Collinsella | Collinsella aerofaciens | AB011814 |
| | | Collinsella sp. CB52 | AB064936 |
| | | Uncultured bacterium clone Eldhufec074 | AY919949 |
| | | Collinsella stercoris | AB031062 |
| | | Collinsella intestinalis | AB031063 |
| | Corynebacterium | Corynebacterium xerosis | AF024653 |
| | | Corynebacterium ulcerans | X81911 |
| | | Corynebacterium ammoniagenes | X82056 |
| | | Corynebacterium pseudodiphtheriticum | X84258 |
| | | uncultured bacterium LI92 | AY978122 |
| | | uncultured bacterium N337 | AY980429 |
| | Eggerthella lenta et rel. | Eggerthella lenta | AB011817 |
| | | uncultured Gram-positive bacterium NO1H5 | AB064862 |
| | | uncultured bacterium ME67 | AY916234 |
| | | Uncultured bacterium clone Eldhufec078 | AY919953 |
| | | Uncultured bacterium clone Eldhufec076 | AY919951 |
| | | Uncultured bacterium clone Eldhufec075 | AY919950 |
| | | Denitrobacterium sp. CCUG 45665 | AJ518870 |
| | | uncultured bacterium Adhufec036abh | AY471677 |
| | Micrococcaceae | Micrococcus luteus | AJ276811 |
| | | Rothia dentocariosa | M59055 |
| | | Uncultured bacterium clone Eldhufec080 | AY919955 |
| | | uncultured bacterium HuJJ72 | AY684419 |
| | Propionibacterium | Propionibacterium acnes | AB041617 |
| | | Propionibacterium avidum | AJ003055 |
| | | Propionibacterium granulosum | AJ003057 |
| | | Propionibacterium propionicum | X53216 |
| | | Propionibacterium jensenii | X53219 |
| | | Propionibacterium acidipropionici | X53221 |
| Bacteroidetes | Alistipes et rel. | Alistipes putredinis | L16497 |
| | | Bacteroides sp. CJ44 | AB080886 |
| | | uncultured bacterium C706 | AY916343 |
| | | uncultured bacterium D080 | AY916354 |
| | | uncultured bacterium M162 | AY916149 |
| | | uncultured bacterium MG06 | AY916286 |
| | | uncultured bacterium NH37 | AY916174 |
| | | uncultured bacterium NN46 | AY916247 |
| | | Uncultured bacterium clone Eldhufec050 | AY919925 |
| | | Uncultured bacterium clone Eldhufec022 | AY919897 |
| | | uncultured bacterium cadhufec076h7 | AF530308 |
| | | uncultured bacterium adhufec52.25 | AF153864 |
| | | Alistipes finegoldii | AJ518874 |
| | | Bacteroides sp. DSM 12148 | AJ518876 |
| | | uncultured bacterium Adhufec002rbh | AY471693 |
| | | Alistipes oderdonkii | AY974072 |
| | | Alistipes shahii | AY974071 |
| | Bacteroides fragilis et rel. | bacterium adhufec23 | AF132251 |
| | | bacterium adhufec355 | AF132263 |
| | | Bacteroides thetaiotaomicron | L16489 |
| | | Bacteroides fragilis | M11656 |
| | | uncultured bacterium MR34 | AY916210 |
| | | uncultured bacterium Z091 | AY916178 |
| | | Uncultured bacterium clone Eldhufec021 | AY919896 |
| | | uncultured bacterium LCRC79 | AF499852 |
| | | Bacteroides finegoldii | AB222699 |
| | | Bacteroides nordii | AY608697 |
| | | Bacteroides salyersiae | AY608696 |
| | Bacteroides intestinalis et rel. | uncultured bacterium OLDA-A11 | AB099761 |
| | | uncultured bacterium HuCA21 | AJ409009 |
| | | Bacteroides intestinalis | AB214329 |
| | Bacteroides ovatus et rel. | Bacteroides ovatus | L16484 |
| | | Bacteroides caccae | X83951 |
| | | uncultured bacterium NC94 | AY916170 |
| | | uncultured bacterium NP35 | AY916253 |
| | | uncultured bacterium HuCA34 | AJ408982 |
| | | uncultured bacterium HuCC30 | AJ315484 |
| | | Uncultured bacterium clone Eldhufec030 | AY919905 |
| | Bacteroides plebeius et rel. | bacterium adhufec367 | AF132266 |
| | | Bacteroides sp. CO11 | AB064922 |
| | | uncultured bacterium D790 | AY916390 |
| | | Uncultured bacterium clone Eldhufec045 | AY919920 |
| | | Uncultured bacterium clone Eldhufec335 | AY920210 |
| | | Bacteroides coprocola | AB200225 |
| | | Bacteroides plebeius | AB200222 |
| | | uncultured bacterium Adhufec025abh | AY471674 |
| | | uncultured bacterium Adhufec086rbh | AY471710 |
| | Bacteroides splachnicus et rel. | bacterium adhufec84 | AF132281 |
| | | Bacteroides splanchnicus | L16496 |
| | | uncultured bacterium C268 | AY916330 |
| | | uncultured bacterium MO48 | AY916145 |
| | | uncultured bacterium MN96 | AY916307 |
| | | uncultured bacterium NK71 | AY916241 |
| | | uncultured bacterium NK90 | AY916243 |
| | | uncultured bacterium NN42 | AY916246 |
| | | uncultured bacterium NN84 | AY916248 |
| | | uncultured bacterium NP53 | AY916254 |
| | | uncultured bacterium NX93 | AY916310 |
| | | Uncultured bacterium clone Eldhufec044 | AY919919 |
| | | Uncultured bacterium clone Eldhufec048 | AY919923 |
| | Bacteroides stercoris et rel. | bacterium adhufec303 | AF132259 |
| | | Bacteroides eggerthii | L16485 |
| | | Bacteroides stercoris | X83953 |
| | | Uncultured bacterium clone Eldhufec057 | AY919932 |
| | | Uncultured bacterium clone Eldhufec025 | AY919900 |
| | Bacteroides uniformis et rel. | Bacteroides uniformis | L16486 |
| | | uncultured Bacteroides sp. NS2A11 | AB064816 |
| | Bacteroides vulgatus et rel. | Bacteroides vulgatus | M58762 |
| | | Bacteroides dorei | AB242142 |
| | Parabacteroides distasonis et | Parabacteroides distasonis | M25249 |
| | rel. | Parabacteroides merdae | X83954 |
| | | uncultured bacterium OLDA-B10 | AB099754 |
| | | uncultured bacterium M270 | AY916152 |
| | | uncultured bacterium MH76 | AY916297 |
| | | Uncultured bacterium clone Eldhufec042 | AY919917 |
| | | uncultured bacterium LCLC20 | AF499837 |
| | | uncultured bacterium ABLCf15 | AF499899 |
| | | Parabacteroides goldsteinii | AY974070 |
| | Prevotella melaninogenica et | bacterium adhufec235 | AF132249 |
| | rel. | Prevotella intermedia | AF414821 |
| | | Prevotella albensis | AJ011683 |
| | | Prevotella melaninogenica | L16469 |
| | | Prevotella veroralis | L16473 |
| | | Prevotella disiens | L16483 |
| | | uncultured bacterium B176 | AY916316 |
| | | uncultured bacterium M107 | AY916148 |
| | | Uncultured bacterium clone Eldhufec008 | AY919883 |
| | | Uncultured bacterium clone Eldhufec007 | AY919882 |
| | | Uncultured bacterium clone Eldhufec033 | AY919908 |
| | | Uncultured bacterium clone Eldhufec038 | AY919913 |
| | | Uncultured bacterium clone Eldhufec037 | AY919912 |
| | | Uncultured bacterium clone Eldhufec036 | AY919911 |
| | | Uncultured bacterium clone Eldhufec035 | AY919910 |
| | | Uncultured bacterium clone Eldhufec034 | AY919909 |
| | | Uncultured bacterium clone Eldhufec005 | AY919880 |
| | | Uncultured bacterium clone Eldhufec009 | AY919884 |
| | | Uncultured bacterium clone Eldhufec024 | AY919899 |
| | | Uncultured bacterium clone Eldhufec019 | AY919894 |
| | | uncultured bacterium HuJJ84 | AY684413 |
| | | Prevotella sp. BI-42 | AJ581354 |
| | Prevotella oralis et rel. | Prevotella oralis | L16480 |
| | | Prevotella sp. CB25 | AB064924 |
| | | uncultured bacterium HuCC28 | AJ315483 |
| | | Uncultured bacterium clone Eldhufec011 | AY919886 |
| | | Uncultured bacterium clone Eldhufec043 | AY919918 |
| | | Uncultured bacterium clone Eldhufec015 | AY919890 |
| | | Uncultured bacterium clone Eldhufec017 | AY919892 |
| | | Uncultured bacterium clone Eldhufec012 | AY919887 |
| | | uncultured bacterium HuJJ29 | AY684415 |
| | | uncultured bacterium Adhufec036rbh | AY471699 |
| | Prevotella ruminicola et rel. | Prevotella ruminicola | AF218618 |
| | | Prevotella brevis | AJ011682 |
| | | Uncultured bacterium clone Eldhufec028 | AY919903 |
| | Prevotella tannerae et rel. | uncultured bacterium OLDC-G2 | AB099769 |
| | | uncultured bacterium OLDC-D5 | AB099768 |
| | | uncultured bacterium ME28 | AY916231 |
| | | Uncultured bacterium clone Eldhufec018 | AY919893 |
| | | Uncultured bacterium clone Eldhufec014 | AY919889 |
| | | Uncultured bacterium clone Eldhufec003 | AY919878 |
| | | uncultured bacterium cadhufec40c10 | AF530373 |
| | Tannerella et rel. | bacterium adhufec77.25 | AF153865 |
| | | uncultured bacterium D487 | AY916372 |
| | | uncultured bacterium D761 | AY916386 |
| | | uncultured bacterium M070 | AY916146 |
| | | uncultured bacterium NG45 | AY916172 |
| | | uncultured bacterium NI77 | AY916176 |
| | | uncultured bacterium NO37 | AY916249 |
| | | uncultured bacterium NO50 | AY916251 |
| | | Uncultured bacterium clone Eldhufec010 | AY919885 |
| | | Uncultured bacterium clone Eldhufec041 | AY919916 |
| | | Uncultured bacterium clone Eldhufec006 | AY919881 |
| | | Uncultured bacterium clone Eldhufec004 | AY919879 |
| | | Uncultured bacterium clone Eldhufec023 | AY919898 |
| | | uncultured bacterium Adhufec048rbh | AY471701 |
| | Unclutured Bacteroidetes | Bacteroides sp. CB40 | AB064919 |
| Asteroleplasma | Asteroleplasma et rel. | Uncultured bacterium UC7-11 | AJ608228 |
| Bacilli | Aerococcus | Aerococcus viridans | M58797 |
| | Bacillus et rel. | Bacillus halodurans | AB013373 |
| | | Bacillus subtilis | AB018484 |
| | | Bacillus pumilus | AB020208 |
| | | Bacillus flexus | AB021185 |
| | | Bacillus cereus | AF076031 |
| | | Bacillus sphaericus | AF169495 |
| | | Brevibacillus brevis | AF424048 |
| | | Bacillus megaterium | D16273 |
| | | Bacillus circulans | D78312 |
| | | Bacillus coagulans | D78313 |
| | | Aneurinibacillus aneurinolyticus | D78455 |
| | | Paenibacillus lautus | D78472 |
| | | Bacillus badius | X77790 |
| | | Paenibacillus durus | X77846 |
| | Enterococcus | Enterococcus faecalis | AB012212 |
| | | Enterococcus faecium | AB012213 |
| | | Enterococcus gallinarum | AF039898 |
| | | Enterococcus casseliflavus | AF039899 |
| | | Enterococcus durans | AF061000 |
| | | Enterococcus avium | AF061008 |
| | | Enterococcus hirae | AF061011 |
| | | uncultured bacterium cadhufec093h7 | AF530310 |
| | | uncultured bacterium (human infant) D8E | AF253331 |
| | Gemella | Gemella morbillorum | L14327 |
| | Granulicatella | Uncultured bacterium clone Eldhufec198 | AY920073 |
| | Lactobacillus gasseri et rel. | Lactobacillus gasseri | AF243142 |
| | | Lactobacillus jensenii | AF243159 |
| | | Lactobacillus crispatus | AF257096 |
| | | Lactobacillus johnsonii | AJ002515 |
| | | Lactobacillus delbrueckii | AY050173 |
| | | Lactobacillus acidophilus | M58802 |
| | | Lactobacillus amylovorus | M58805 |
| | | Lactobacillus helveticus | X61141 |
| | | uncultured Lactobacillus sp. LabF368 | AF335876 |
| | | uncultured Lactobacillus sp. LabF93 | AF335911 |
| | | Lactobacillus ultunensis | AY253660 |
| | | Lactobacillus kalixensis | AY253657 |
| | Lactobacillus plantarum et rel. | Pediococcus acidilactici | AB018213 |
| | | Lactobacillus brevis | AB024299 |
| | | Lactobacillus mucosae | AF126738 |
| | | Lactobacillus rhamnosus | AF243146 |
| | | Lactobacillus paracasei | AF243147 |
| | | Lactobacillus fermentum | AF243149 |
| | | Lactobacillus vaginalis | AF243177 |
| | | Lactobacillus plantarum | AJ271852 |
| | | Lactobacillus casei | AJ272201 |
| | | Lactobacillus pentosus | D79211 |
| | | Lactobacillus reuteri | L23507 |
| | | Lactobacillus buchneri | M58811 |
| | | Pediococcus pentosaceus | M58834 |
| | | Lactobacillus oris | X61131 |
| | | uncultured Lactobacillus sp. LabS14 | AF335913 |
| | | Lactobacillus antri | AY253659 |
| | | Lactobacillus gastricus | AY253658 |
| | | Lactobacillus parabuchneri | AB205056 |
| | Lactobacillus sakei et rel. | Lactobacillus sakei | M58829 |
| | Lactobacillus salivarius et rel. | Lactobacillus salivarius | AF420311 |
| | | Lactobacillus ruminis | M58828 |
| | Lactococcus | Lactococcus lactis | AJ271851 |
| | | Lactococcus sp. 451 | AY762109 |
| | Staphylococcus | Staphylococcus aureus | AF015929 |
| | | Staphylococcus epidermidis | D83362 |
| | | Staphylococcus saccharolyticus | L37602 |
| | Streptococcus bovis et rel. | Streptococcus equinus | AB002514 |
| | | Streptococcus uberis | AB023573 |
| | | Streptococcus agalactiae | AB023574 |
| | | Streptococcus pyogenes | AF076028 |
| | | Streptococcus bovis | AF104109 |
| | | Streptococcus infantarius | AF177729 |
| | | Streptococcus lutetiensis | AF429763 |
| | | Streptococcus salivarius | M58839 |
| | | Streptococcus thermophilus | X59028 |
| | | uncultured bacterium OLDA-B7 | AB099789 |
| | | Streptococcus equi subsp. zooepidemicus | AB104843 |
| | | Streptococcus equisimilis | AJ314611 |
| | Streptococcus intermedius et | Streptococcus intermedius | AF104671 |
| | rel. | Streptococcus constellatus | AF104676 |
| | | Streptococcus anginosus | AF145240 |
| | | Streptococcus parasanguinis | X53652 |
| | | Uncultured bacterium clone Eldhufec195 | AY920070 |
| | Streptococcus mitis et rel. | Streptococcus sanguis | AF003928 |
| | | Streptococcus mitis | AF003929 |
| | | Streptococcus oralis | AF003932 |
| | | Streptococcus viridans | AF076036 |
| | | Streptococcus mutans | AJ243965 |
| | | uncultured Streptococcus sp. NB5C1 | AB064839 |
| | | bacterium ucfecDB2 | ARB_B5C8DA |
| | Weissella et rel. | Weissella cibaria | AJ295989 |
| | | Leuconostoc mesenteroides | M23035 |
| | | Weissella confusa | M23036 |
| | | uncultured Leuconostoc sp. LabF165 | AF335897 |
| Clostridium | Clostridium | Eubacterium multiforme | AB018184 |
| cluster I | | Clostridium paraputrificum | AB032556 |
| | | Clostridium perfringens | AB045282 |
| | | Clostridium botulinum | AF105402 |
| | | Sarcina ventriculi | AF110272 |
| | | Clostridium putrefaciens | AF127024 |
| | | Clostridium subterminale | AF241842 |
| | | Clostridium butyricum | AJ002592 |
| | | Clostridium tertium | AJ245413 |
| | | Clostridium tyrobutyricum | L08062 |
| | | Eubacterium moniliforme | L34622 |
| | | Clostridium cadaveris | M59086 |
| | | Clostridium fallax | M59088 |
| | | Clostridium cochlearium | M59093 |
| | | Clostridium limosum | M59096 |
| | | Clostridium malenominatum | M59099 |
| | | Clostridium paraperfringens | M59102 |
| | | Clostridium sporogenes | M59115 |
| | | Clostridium acetobutylicum | S46735 |
| | | Clostridium septicum | U59278 |
| | | Clostridium barati | X68174 |
| | | Clostridium beijerinckii | X68179 |
| | | Clostridium celatum | X77844 |
| | | Clostridium sartagoformum | Y18175 |
| | | Uncultured bacterium clone Eldhufec341 | AY920216 |
| | | Eubacterium budayi | AB018183 |
| | | Eubacterium nitritogenes | AB018185 |
| Clostridium | Clostridium stercorarium et | uncultured bacterium B839 | AY916322 |
| cluster III | rel. | uncultured bacterium D145 | AY916358 |
| | | uncultured bacterium LE17 | AY916205 |
| | | Uncultured bacterium clone Eldhufec339 | AY920214 |
| | | Uncultured bacterium UC7-82 | AJ608246 |
| | Clostridium thermocellum et | uncultured bacterium C288 | AY916331 |
| | rel. | Uncultured bacterium clone Eldhufec338 | AY920213 |
| Clostridium | Anaerotruncus colihominis et | bacterium adhufec101 | AF132235 |
| cluster IV | rel. | uncultured Gram-positive bacterium NO2-2 | AB064805 |
| | | uncultured bacterium D577 | AY916375 |
| | | uncultured bacterium LF02 | AY916207 |
| | | uncultured bacterium LL29 | AY916260 |
| | | uncultured bacterium LL87 | AY916261 |
| | | uncultured bacterium HuCA1 | AJ408957 |
| | | Uncultured bacterium clone Eldhufec246 | AY920121 |
| | | Uncultured bacterium clone Eldhufec211 | AY920086 |
| | | Uncultured bacterium clone Eldhufec214 | AY920089 |
| | | Uncultured bacterium clone Eldhufec215 | AY920090 |
| | | Uncultured bacterium clone Eldhufec265 | AY920140 |
| | | Uncultured bacterium clone Eldhufec270 | AY920145 |
| | | Anaerotruncus colihominis | AJ315980 |
| | Clostridium cellulosi rel. | uncultured human gut bacterium JW1B12 | AB080849 |
| | | uncultured bacterium OLDB-E4 | AB099734 |
| | | uncultured bacterium C342 | AY916333 |
| | | uncultured bacterium D036 | AY916351 |
| | | uncultured bacterium K507 | AY916200 |
| | | uncultured bacterium LZ45 | AY916188 |
| | | uncultured bacterium M490 | AY916159 |
| | | uncultured bacterium M511 | AY916162 |
| | | uncultured bacterium MH24 | AY916292 |
| | | uncultured bacterium Z456 | AY916179 |
| | | uncultured bacterium D626 | AY916378 |
| | | Uncultured bacterium clone Eldhufec236 | AY920111 |
| | | Uncultured bacterium clone Eldhufec212 | AY920087 |
| | | Uncultured bacterium clone Eldhufec213 | AY920088 |
| | | Uncultured bacterium clone Eldhufec273 | AY920148 |
| | | Uncultured bacterium clone Eldhufec249 | AY920124 |
| | | Uncultured bacterium UC7-44 | AJ608241 |
| | | Uncultured bacterium UC7-69 | AJ608244 |
| | | uncultured bacterium cadhufec022h7 | AF530299 |
| | | uncultured bacterium ABLCf36 | AF499903 |
| | | uncultured bacterium HuAC35 | AY684394 |
| | | uncultured bacterium Adhufec106abh | AY471691 |
| | Clostridium leptum et rel. | Clostridium leptum | M59095 |
| | | Clostridium sporosphaeroides | M59116 |
| | | uncultured human gut bacterium JW1C7 | AB080848 |
| | | uncultured bacterium C464 | AY916336 |
| | | uncultured bacterium C735 | AY916345 |
| | | uncultured bacterium K288 | AY916193 |
| | | uncultured bacterium HuCA24 | AJ408976 |
| | | Uncultured bacterium clone Eldhufec221 | AY920096 |
| | | Uncultured bacterium UC7-14 | AJ608230 |
| | | uncultured bacterium adhufec168 | AF132242 |
| | | Ruminococcus sp. 16442 | AJ318889 |
| | Clostridium orbiscindens et | Clostridium orbiscindens | Y18187 |
| | rel. | human intestinal firmicute CJ36 | AB080896 |
| | | human intestinal firmicute CJ31 | AB080897 |
| | | uncultured human gut bacterium JW1D6 | AB080858 |
| | | uncultured human gut bacterium JW2G1 | AB080857 |
| | | uncultured human gut bacterium JW1G9 | AB080856 |
| | | uncultured human gut bacterium JW2A8 | AB080855 |
| | | uncultured bacterium OLDA-F4 | AB099727 |
| | | uncultured bacterium B632 | AY916320 |
| | | uncultured bacterium D330 | AY916365 |
| | | uncultured bacterium D465 | AY916371 |
| | | uncultured bacterium D588 | AY916376 |
| | | uncultured bacterium G267 | AY916285 |
| | | uncultured bacterium K351 | AY916196 |
| | | uncultured bacterium LV67 | AY916184 |
| | | uncultured bacterium M510 | AY916161 |
| | | uncultured bacterium W074 | AY916213 |
| | | uncultured bacterium HuCB24 | AJ408998 |
| | | Uncultured bacterium clone Eldhufec218 | AY920093 |
| | | Uncultured bacterium clone Eldhufec272 | AY920147 |
| | | Uncultured bacterium clone Eldhufec262 | AY920137 |
| | | Uncultured bacterium clone Eldhufec264 | AY920139 |
| | | Uncultured bacterium clone Eldhufec267 | AY920142 |
| | | Uncultured bacterium clone Eldhufec229 | AY920104 |
| | | uncultured bacterium cadhufec074h7 | AF530307 |
| | | Bacteroides capillosus | AY136666 |
| | | uncultured bacterium Adhufec102rbh | AY471712 |
| | Eubacterium siraeum et rel. | Eubacterium siraeum | L34625 |
| | | uncultured bacterium B025 | AY916313 |
| | | Uncultured bacterium clone Eldhufec237 | AY920112 |
| | | Uncultured bacterium clone Eldhufec239 | AY920114 |
| | | Uncultured bacterium UC7-117 | AJ608247 |
| | | uncultured bacterium Adhufec058abh | AY471683 |
| | Faecalibacterium prausnitzii | bacterium adhufec113 | AF132236 |
| | et rel. | butyrate-producing bacterium A2-165 | AJ270469 |
| | | butyrate-producing bacterium L2-6 | AJ270470 |
| | | Faecalibacterium prausnitzii | AJ413954 |
| | | uncultured bacterium KM82 | AY916180 |
| | | uncultured bacterium KP66 | AY916136 |
| | | uncultured bacterium HuCA25 | AJ408973 |
| | | uncultured bacterium HuCA11 | AJ408966 |
| | | Uncultured bacterium clone Eldhufec238 | AY920113 |
| | | Uncultured bacterium clone Eldhufec226 | AY920101 |
| | | Uncultured bacterium clone Eldhufec227 | AY920102 |
| | | Uncultured bacterium clone Eldhufec288 | AY920163 |
| | | Uncultured bacterium clone Eldhufec228 | AY920103 |
| | | Uncultured bacterium clone Eldhufec259 | AY920134 |
| | | Uncultured bacterium clone Eldhufec261 | AY920136 |
| | | Uncultured bacterium clone Eldhufec276 | AY920151 |
| | | Uncultured bacterium clone Eldhufec282 | AY920157 |
| | | Uncultured bacterium clone Eldhufec256 | AY920131 |
| | | Uncultured bacterium clone Eldhufec255 | AY920130 |
| | | Uncultured bacterium clone Eldhufec252 | AY920127 |
| | | Uncultured bacterium clone Eldhufec281 | AY920156 |
| | | Uncultured bacterium clone Eldhufec251 | AY920126 |
| | | uncultured bacterium adhufec08.25 | AF153871 |
| | | uncultured bacterium A10 | AF052411 |
| | | uncultured bacterium Adhufec010abh | AY471671 |
| | | uncultured bacterium Adhufec055abh | AY471682 |
| | | uncultured bacterium Adhufec052abh | AY471681 |
| | | uncultured bacterium Adhufec064rbh | AY471704 |
| | | uncultured bacterium Adhufec057rbh | AY471702 |
| | | uncultured bacterium Adhufec107rbh | AY471714 |
| | Oscillospira guillermondii et | bacterium adhufec269 | AF132255 |
| | rel. | uncultured human gut bacterium JW1C11 | AB080854 |
| | | uncultured bacterium OLDA-D11 | AB099726 |
| | | uncultured bacterium OLDC-D12 | AB099725 |
| | | uncultured bacterium OLDA-H2 | AB099721 |
| | | uncultured bacterium A051 | AY916256 |
| | | uncultured bacterium B811 | AY916321 |
| | | uncultured bacterium C574 | AY916337 |
| | | uncultured bacterium D134 | AY916357 |
| | | uncultured bacterium D288 | AY916364 |
| | | uncultured bacterium D440 | AY916370 |
| | | uncultured bacterium LE02 | AY916204 |
| | | uncultured bacterium MA30 | AY916224 |
| | | uncultured bacterium MM71 | AY916303 |
| | | uncultured bacterium V239 | AY916276 |
| | | uncultured bacterium HuCB7 | AJ408991 |
| | | Uncultured bacterium clone Eldhufec241 | AY920116 |
| | | Uncultured bacterium clone Eldhufec223 | AY920098 |
| | | Uncultured bacterium clone Eldhufec257 | AY920132 |
| | | Uncultured bacterium clone Eldhufec301 | AY920176 |
| | | Uncultured bacterium clone Eldhufec285 | AY920160 |
| | | Uncultured bacterium clone Eldhufec283 | AY920158 |
| | | uncultured bacterium cadhufec121h7 | AF530315 |
| | | uncultured bacterium Adhufec002abh | AY471669 |
| | | uncultured bacterium Adhufec044abh | AY471679 |
| | Outgrouping Clostridium | uncultured bacterium C747 | AY916347 |
| | cluster IV | uncultured bacterium LD25 | AY916202 |
| | | uncultured bacterium V366 | AY916279 |
| | | Uncultured bacterium clone Eldhufec318 | AY920193 |
| | | Uncultured bacterium clone Eldhufec320 | AY920195 |
| | | Uncultured bacterium clone Eldhufec321 | AY920196 |
| | | Uncultured bacterium clone Eldhufec319 | AY920194 |
| | Papillibacter cinnamivorans et | bacterium adhufec296 | AF132258 |
| | rel. | butyrate-producing bacterium A2-207 | AJ270471 |
| | | uncultured Gram-positive bacterium NB5F9 | AB064783 |
| | | uncultured bacterium ZO15 | AY916177 |
| | | Uncultured bacterium clone Eldhufec233 | AY920108 |
| | | Uncultured bacterium clone Eldhufec245 | AY920120 |
| | | Uncultured bacterium clone Eldhufec258 | AY920133 |
| | | uncultured bacterium cadhufec32c10 | AF530372 |
| | Ruminococcus bromii et rel. | Ruminococcus bromii | L76600 |
| | | uncultured bacterium HuCB2 | AJ408987 |
| | | Uncultured bacterium clone Eldhufec230 | AY920105 |
| | | Uncultured bacterium clone Eldhufec291 | AY920166 |
| | | Uncultured bacterium clone Eldhufec225 | AY920100 |
| | | Uncultured bacterium clone Eldhufec291 | AY920166 |
| | | uncultured bacterium cadhufec021h7 | AF530298 |
| | | uncultured bacterium Adhufec014rbh | AY471694 |
| | Ruminococcus callidus et rel. | Ruminococcus flavefaciens | AF030446 |
| | | Ruminococcus albus | AF030451 |
| | | Ruminococcus callidus | L76596 |
| | | Clostridium methylpentosum | Y18181 |
| | | uncultured Gram-positive bacterium NS4G9 | AB064811 |
| | | uncultured Ruminococcus sp. NO11 | AB064808 |
| | | uncultured bacterium D005 | AY916350 |
| | | uncultured bacterium D739 | AY916385 |
| | | uncultured bacterium D789 | AY916389 |
| | | uncultured bacterium MF20 | AY916235 |
| | | uncultured bacterium MH26 | AY916293 |
| | | Uncultured bacterium clone Eldhufec235 | AY920110 |
| | | Uncultured bacterium clone Eldhufec284 | AY920159 |
| | | Uncultured bacterium clone Eldhufec250 | AY920125 |
| | Sporobacter termitidis rel. | bacterium adhufec311 | AF132261 |
| | | bacterium adhufec108 | AF132283 |
| | | uncultured bacterium OLDC-E8 | AB099728 |
| | | uncultured bacterium C352 | AY916334 |
| | | uncultured bacterium C354 | AY916335 |
| | | uncultured bacterium C727 | AY916344 |
| | | uncultured bacterium D762 | AY916387 |
| | | uncultured bacterium L495 | AY916281 |
| | | uncultured bacterium LO41 | AY916265 |
| | | uncultured bacterium LQ71 | AY916268 |
| | | uncultured bacterium LY18 | AY916187 |
| | | Uncultured bacterium clone Eldhufec210 | AY920085 |
| | | Uncultured bacterium clone Eldhufec290 | AY920165 |
| | | Uncultured bacterium clone Eldhufec274 | AY920149 |
| | | Uncultured bacterium clone Eldhufec231 | AY920106 |
| | | Uncultured bacterium clone Eldhufec294 | AY920169 |
| | | Uncultured bacterium clone Eldhufec216 | AY920091 |
| | | Uncultured bacterium clone Eldhufec217 | AY920092 |
| | | Uncultured bacterium clone Eldhufec287 | AY920162 |
| | | Uncultured bacterium clone Eldhufec220 | AY920095 |
| | | Uncultured bacterium clone Eldhufec232 | AY920107 |
| | | Uncultured bacterium UC7-1 | AJ608220 |
| | Subdoligranulum variable at | bacterium adhufec13 | AF132237 |
| | rel. | uncultured Gram-positive bacterium NO2- | AB064804 |
| | | uncultured Gram-positive bacterium NB5C6 | AB064803 |
| | | human intestinal firmicute CJ7 | AB080895 |
| | | uncultured human gut bacterium JW1D4 | AB080847 |
| | | uncultured bacterium LC79 | AY916201 |
| | | uncultured bacterium M479 | AY916158 |
| | | uncultured bacterium HuCB5 | AJ408989 |
| | | Uncultured bacterium clone Eldhufec243 | AY920118 |
| | | Uncultured bacterium clone Eldhufec222 | AY920097 |
| | | Uncultured bacterium clone Eldhufec224 | AY920099 |
| | | Uncultured bacterium clone Eldhufec260 | AY920135 |
| | | Uncultured bacterium clone Eldhufec302 | AY920177 |
| | | Uncultured bacterium clone Eldhufec268 | AY920143 |
| | | uncultured bacterium cadhufec068h7 | AF530306 |
| | | uncultured bacterium cadhufec066h7 | AF530305 |
| | | uncultured bacterium ABLCf22 | AF499901 |
| | | Subdoligranulum variabile | AJ518869 |
| Clostridium | Dialister | Dialister pneumosintes | X82500 |
| cluster IX | | uncultured Gram-positive bacterium NS2B1 | AB064859 |
| | | Uncultured bacterium clone Eldhufec091 | AY919966 |
| | | Uncultured bacterium clone Eldhufec093 | AY919968 |
| | | Uncultured bacterium clone Eldhufec089 | AY919964 |
| | | Uncultured bacterium clone Eldhufec096 | AY919971 |
| | | uncultured bacterium B856 | AY984881 |
| | | uncultured bacterium MG10 | AY982155 |
| | Megamonas hypermegale et | Megamonas hypermegale | AJ420107 |
| | rel. | human intestinal firmicute CB15 | AB064931 |
| | | uncultured bacterium cadhufec43c10 | AF530374 |
| | Megasphaera elsdenii et rel. | Megasphaera elsdenii | AF283705 |
| | | uncultured bacterium OLDC-D10 | AB099774 |
| | | uncultured bacterium HuCB85 | AJ409007 |
| | | Uncultured bacterium clone Eldhufec098 | AY919973 |
| | | uncultured bacterium inhufecA-11 | AY328359 |
| | Mitsuokella multiacida et rel. | Selenomonas ruminantium | AB017195 |
| | | Mitsuokella multiacida | X81878 |
| | | uncultured Gram-positive bacterium NB5E1 | AB064853 |
| | | uncultured bacterium OLDC-C6 | AB099772 |
| | Peptococcus niger et rel. | Peptococcus niger | X55797 |
| | | uncultured bacterium D393 | AY916367 |
| | | uncultured bacterium MH31 | AY916294 |
| | | uncultured bacterium V247 | AY916277 |
| | | Uncultured bacterium clone Eldhufec095 | AY919970 |
| | | uncultured bacterium HuDI10 | AY862394 |
| | Phascolarctobacterium | bacterium adhufec395 | AF132234 |
| | faecium et rel. | Acidaminococcus fermentans | X65935 |
| | | uncultured Gram-positive bacterium NB4G9 | AB064849 |
| | | uncultured bacterium OLDB-D6 | AB099771 |
| | | uncultured bacterium OLDB-B2 | AB099753 |
| | | uncultured bacterium D115 | AY916356 |
| | | Uncultured bacterium clone Eldhufec097 | AY919972 |
| | | Uncultured bacterium clone Eldhufec094 | AY919969 |
| | | uncultured bacterium cadhufec137c10 | AF530370 |
| | Uncultured Selenomonadaceae | uncultured bacterium HuAC20 | AY684401 |
| | Veillonella | Veillonella dispar | AF439639 |
| | | Veillonella parvula | AF439640 |
| | | Veillonella atypica | AF439641 |
| | | uncultured bacterium ABLCf8 | AF499900 |
| Clostridium | Anaerovorax odorimutans rel. | uncultured Gram-positive bacterium NO2-6 | AB064863 |
| cluster XI | | uncultured human gut bacterium JW1G2 | AB080883 |
| | | uncultured bacterium LN56 | AY916263 |
| | | uncultured bacterium MO17 | AY916142 |
| | | uncultured bacterium MH36 | AY916295 |
| | | uncultured bacterium P615 | AY916312 |
| | | Uncultured bacterium clone Eldhufec185 | AY920060 |
| | | Uncultured bacterium clone Eldhufec187 | AY920062 |
| | | Uncultured bacterium clone Eldhufec186 | AY920061 |
| | | uncultured bacterium HuJJ43 | AY684403 |
| | | uncultured bacterium HuRC86 | AY684402 |
| | Clostridium difficile et rel. | Clostridium hiranonis | AB023970 |
| | | Clostridium difficile | AF072473 |
| | | Clostridium bifermentans | AF320283 |
| | | Clostridium glycolicum | AY007244 |
| | | Clostridium sticklandii | L04167 |
| | | Clostridium sordellii | M59105 |
| | | Eubacterium tenue | M59118 |
| | | Clostridium irregularis | X73447 |
| | | Clostridium ghoni | X73451 |
| | | uncultured Gram-positive bacterium NS1E9 | AB064876 |
| | | uncultured Clostridium sp. NB4D7 | AB064872 |
| | | uncultured bacterium OLDB-G12 | AB099796 |
| | | uncultured bacterium M364 | AY916153 |
| | | Uncultured bacterium clone Eldhufec189 | AY920064 |
| | | uncultured bacterium LCLC73 | AF499844 |
| | | uncultured bacterium LCLC21 | AF499843 |
| | | Clostridium bartlettii | AY438672 |
| | Clostridium felsineum | Clostridium felsineum | X77851 |
| | Peptostreptococcus | Peptostreptococcus anaerobius | D14150 |
| | anaerobius et rel. | uncultured bacterium C120 | AY916327 |
| Clostridium | Peptostreptococcus micros et | Peptoniphilus asaccharolyticus | D14138 |
| cluster XIII | rel. | Anaerococcus prevotii | D14139 |
| | | Anaerococcus hydrogenalis | D14140 |
| | | Peptostreptococcus micros | D14143 |
| | | Peptoniphilus indolicus | D14147 |
| | | Finegoldia magna | D14149 |
| | | uncultured bacterium G170 | AY981208 |
| | Tissierella | Tissierella praeacuta | X80833 |
| Clostridium | Acetitomaculum ruminis rel. | bacterium adhufec250 | AF132253 |
| cluster XIVa | | uncultured bacterium D416 | AY916368 |
| | | uncultured bacterium LP40 | AY916266 |
| | | uncultured bacterium M977 | AY916221 |
| | | Uncultured bacterium clone Eldhufec157 | AY920032 |
| | | Uncultured bacterium clone Eldhufec120 | AY919995 |
| | | Uncultured bacterium clone Eldhufec117 | AY919992 |
| | | Uncultured bacterium clone Eldhufec110 | AY919985 |
| | | Uncultured bacterium clone Eldhufec103 | AY919978 |
| | | uncultured bacterium HuDI84 | AY684365 |
| | Anaerostipes caccae et rel. | Clostridium indolis | AF028351 |
| | | bacterium adhufec25 | AF132254 |
| | | Anaerostipes caccae | AJ270487 |
| | | uncultured Gram-positive bacterium NB2G8 | AB064714 |
| | | uncultured Gram-positive bacterium NO2-5 | AB064713 |
| | | uncultured human gut bacterium JW2C7 | AB080875 |
| | | uncultured bacterium HuCA20 | AJ408972 |
| | Bryantella formatexigens et | bacterium adhufec40 | AF132270 |
| | rel. | Eubacterium cellulosolvens | L34613 |
| | | uncultured Gram-positive bacterium NS2F9 | AB064773 |
| | | Ruminococcus sp. CO28 | AB064891 |
| | | uncultured bacterium M629 | AY916166 |
| | | uncultured bacterium M963 | AY916220 |
| | | uncultured bacterium ME57 | AY916233 |
| | | uncultured bacterium MF29 | AY916238 |
| | | uncultured bacterium P315 | AY916311 |
| | | Uncultured bacterium clone Eldhufec135 | AY920010 |
| | | Uncultured bacterium clone Eldhufec152 | AY920027 |
| | | Uncultured bacterium UC7-3 | AJ608221 |
| | | Uncultured bacterium UC7-50 | AJ608242 |
| | | uncultured bacterium cadhufec56c10 | AF530376 |
| | | uncultured bacterium ABLCf44 | AF499907 |
| | | Bryantella formatexigens | AJ318527 |
| | | uncultured bacterium HuRC75 | AY684376 |
| | | uncultured bacterium Adhufec124abh | AY471692 |
| | Butyrivibrio crossotus et rel. | bacterium adhufec406 | AF132269 |
| | | Eubacterium ramulus | AJ011522 |
| | | Butyrivibrio crossotus | X89981 |
| | | uncultured bacterium D680 | AY916379 |
| | | uncultured bacterium D692 | AY916380 |
| | | uncultured bacterium D726 | AY916383 |
| | | uncultured bacterium D738 | AY916384 |
| | | uncultured bacterium MG71 | AY916289 |
| | | Uncultured bacterium clone Eldhufec138 | AY920013 |
| | | Uncultured bacterium clone Eldhufec155 | AY920030 |
| | | Uncultured bacterium clone Eldhufec116 | AY919991 |
| | | Uncultured bacterium clone Eldhufec114 | AY919989 |
| | | Uncultured bacterium clone Eldhufec112 | AY919987 |
| | | Uncultured bacterium clone Eldhufec147 | AY920022 |
| | | Uncultured bacterium clone Eldhufec244 | AY920119 |
| | | uncultured bacterium Adhufec023abh | AY471673 |
| | | uncultured bacterium Adhufec112rbh | AY471715 |
| | | uncultured bacterium Muc3-1 | AY451999 |
| | Clostridium | uncultured human gut bacterium JW1G3 | AB080863 |
| | glycyrrhizinilyticum et rel. | uncultured human gut bacterium JW1A12 | AB080860 |
| | | uncultured bacterium NP09 | AY916252 |
| | | uncultured bacterium HuCC43 | AJ315487 |
| | | Uncultured bacterium clone Eldhufec125 | AY920000 |
| | | Uncultured bacterium clone Eldhufec123 | AY919998 |
| | | uncultured bacterium cadhufec69c10 | AF530380 |
| | | uncultured bacterium cadhufec101h7 | AF530314 |
| | | uncultured bacterium HuRC12 | AY684370 |
| | | Clostridium glycyrrhizinilyticum | AB233029 |
| | Clostridium lactifermentans et | uncultured bacterium G075 | AY916283 |
| | rel. | uncultured bacterium K305 | AY916194 |
| | | uncultured bacterium NK21 | AY916240 |
| | | Uncultured bacterium clone Eldhufec141 | AY920016 |
| | | Uncultured bacterium clone Eldhufec182 | AY920057 |
| | | Uncultured bacterium clone Eldhufec183 | AY920058 |
| | | uncultured bacterium HuDI72 | AY684405 |
| | | uncultured bacterium HuDI23 | AY684406 |
| | | Clostridium lactatifermentans | AY033434 |
| | Clostridium nexile et rel. | butyrate-producing bacterium A2-231 | AJ270484 |
| | | Clostridium nexile | X73443 |
| | | uncultured Gram-positive bacterium NB4C3 | AB064747 |
| | | uncultured Gram-positive bacterium NO2-4 | AB064746 |
| | | uncultured Gram-positive bacterium NO31 | AB064743 |
| | | uncultured Gram-positive bacterium NO81 | AB064742 |
| | | uncultured bacterium OLDB-F3 | AB099735 |
| | | uncultured bacterium cadhufec20a04 | AF530331 |
| | | uncultured bacterium LCRC24 | AF499855 |
| | | uncultured bacterium ABLC1 | AF499881 |
| | | uncultured bacterium ABLCf89 | AF499909 |
| | Clostridium sphenoides et rel. | bacterium A21 | AF052418 |
| | | bacterium A54 | AF052421 |
| | | bacterium adhufec382 | AF132267 |
| | | Clostridium sphenoides | X73449 |
| | | uncultured Gram-positive bacterium NB2A8 | AB064730 |
| | | uncultured Gram-positive bacterium NO2-2 | AB064727 |
| | | uncultured bacterium HuCA27 | AJ408978 |
| | | uncultured bacterium HuCA19 | AJ408971 |
| | | uncultured bacterium HuCA17 | AJ408969 |
| | | uncultured bacterium LCLC63 | AF499839 |
| | | uncultured bacterium LCLC23 | AF499838 |
| | | uncultured bacterium ABLC30 | AF499880 |
| | | uncultured bacterium ABLCf11 | AF499906 |
| | | Clostridium hathewayi | AJ311620 |
| | | uncultured bacterium Adhufec088khh | AY471662 |
| | Clostridium symbiosum et rel. | Clostridium clostridiiformes | M59089 |
| | | Clostridium symbiosum | M59112 |
| | | Clostridium sp. CJ23 | AB080893 |
| | | uncultured bacterium B147 | AY916315 |
| | | uncultured bacterium B395 | AY916317 |
| | | uncultured bacterium B840 | AY916323 |
| | | uncultured bacterium K375 | AY916197 |
| | | uncultured bacterium L812 | AY916282 |
| | | uncultured bacterium MB66 | AY916225 |
| | | uncultured bacterium MD61 | AY916228 |
| | | uncultured bacterium MI29 | AY916299 |
| | | uncultured bacterium HuCC34 | AJ315486 |
| | | Uncultured bacterium clone Eldhufec149 | AY920024 |
| | | Uncultured bacterium clone Eldhufec115 | AY919990 |
| | | Uncultured bacterium clone Eldhufec100 | AY919975 |
| | | uncultured bacterium inhufecA-32 | AY328366 |
| | | uncultured bacterium LCTI22 | AF499870 |
| | | Clostridium asparagiforme | AJ582080 |
| | | Clostridium bolteae | AJ508452 |
| | | butyrate-producing bacterium M62/1 | AY305309 |
| | | uncultured bacterium M985 | AY983861 |
| | Coprococcus catus et rel. | butyrate-producing bacterium L2-10 | AJ270486 |
| | | uncultured human gut bacterium JW1B8 | AB080861 |
| | | uncultured bacterium KO89 | AY916135 |
| | | uncultured bacterium NW71 | AY916309 |
| | | Uncultured bacterium UC7-62 | AJ608243 |
| | | uncultured bacterium cadhufec098h7 | AF530312 |
| | | Coprococcus catus | AB038359 |
| | Coprococcus eutactus et rel. | Eubacterium ruminantium | AB008552 |
| | | bacterium A57 | AF052422 |
| | | bacterium adhufec157 | AF132241 |
| | | butyrate-producing bacterium A2-166 | AJ270489 |
| | | Coprococcus eutactus | D14148 |
| | | uncultured Ruminococcus sp. NB2B8 | AB064761 |
| | | Uncultured bacterium UC7-8 | AJ608226 |
| | Dorea formicigenerans et rel. | Clostridium scindens | AB020727 |
| | | Clostridium hylemonae | AB023972 |
| | | bacterium A71 | AF052423 |
| | | Dorea formicigenerans | L34619 |
| | | uncultured Gram-positive bacterium NS2C1 | AB064738 |
| | | human intestinal firmicute CO39 | AB064889 |
| | | uncultured human gut bacterium JW1H4b | AB080873 |
| | | uncultured bacterium KW79 | AY916215 |
| | | uncultured bacterium N874 | AY916190 |
| | | uncultured bacterium HuCB21 | AJ408996 |
| | | Dorea longicatena | AJ132842 |
| | Eubacterium hallii et rel. | Eubacterium hallii | L34621 |
| | | uncultured bacterium HuCB26 | AJ409000 |
| | | uncultured bacterium HuCC15 | AJ315482 |
| | | uncultured bacterium Adhufec106khh | AY471665 |
| | | uncultured bacterium Adhufec127rbh | AY471720 |
| | | bacterium ucfecDC6 | |
| | Eubacterium rectale et rel. | Butyrivibrio fibrisolvens | AB004910 |
| | | Eubacterium rectale | L34627 |
| | | uncultured bacterium D522 | AY916373 |
| | | uncultured bacterium M372 | AY916154 |
| | | uncultured bacterium HuCB37 | AJ409004 |
| | | uncultured bacterium HuCA8 | AJ408964 |
| | | Uncultured bacterium clone Eldhufec130 | AY920005 |
| | | Uncultured bacterium clone Eldhufec121 | AY919996 |
| | | Lachnobacterium sp. wal 14165 | AJ518873 |
| | | uncultured bacterium A22 | AF052419 |
| | Eubacterium ventriosum et rel. | bacterium adhufec335 | AF132262 |
| | | Eubacterium ventriosum | L34421 |
| | | uncultured bacterium D177 | AY916360 |
| | Lachnobacillus bovis et rel. | bacterium A11 | AF052412 |
| | | bacterium adhufec68 | AF132278 |
| | | uncultured bacterium B558 | AY916318 |
| | | uncultured bacterium D695 | AY916382 |
| | | uncultured bacterium ME11 | AY916230 |
| | | Uncultured bacterium clone Eldhufec139 | AY920014 |
| | | Uncultured bacterium clone Eldhufec137 | AY920012 |
| | | Uncultured bacterium clone Eldhufec153 | AY920028 |
| | | Uncultured bacterium clone Eldhufec118 | AY919993 |
| | Lachnospira pectinoschiza et | Lachnospira pectinoschiza | L14675 |
| | rel. | Eubacterium eligens | L34420 |
| | | uncultured bacterium LZ58 | AY916189 |
| | | Uncultured bacterium clone Eldhufec140 | AY920015 |
| | | Uncultured bacterium clone Eldhufec105 | AY919980 |
| | | Uncultured bacterium UC7-131 | AJ608250 |
| | | uncultured bacterium ABLCf6 | AF499905 |
| | Outgrouping Clostridium | bacterium adhufec236 | AF132250 |
| | cluster XIVa | bacterium adhufec295 | AF132257 |
| | | bacterium adhufec405 | AF132268 |
| | | bacterium adhufec52 | AF132274 |
| | | Clostridium aminovalericum | M23929 |
| | | uncultured human gut bacterium JW1C1 | AB080872 |
| | | uncultured human gut bacterium JW1D8 | AB080871 |
| | | uncultured bacterium LL95 | AY916262 |
| | | uncultured bacterium MK42 | AY916301 |
| | | uncultured bacterium N322 | AY916273 |
| | | uncultured bacterium NL43 | AY916244 |
| | | uncultured bacterium V213 | AY916275 |
| | | uncultured bacterium HuCB56 | AJ409006 |
| | | Uncultured bacterium clone Eldhufec129 | AY920004 |
| | | Uncultured bacterium clone Eldhufec184 | AY920059 |
| | | Uncultured bacterium clone Eldhufec111 | AY919986 |
| | | butyrate-producing bacterium SS3/4 | AY305316 |
| | | uncultured bacterium HuAC36 | AY684386 |
| | | uncultured bacterium Adhufec004abh | AY471670 |
| | | uncultured bacterium Adhufec071rbh | AY471707 |
| | | uncultured bacterium Muc3-13 | AY452004 |
| | Roseburia intestinalis et rel. | butyrate-producing bacterium A2-183 | AJ270482 |
| | | Uncultured bacterium clone Eldhufec122 | AY919997 |
| | | butyrate-producing bacterium M72/1 | AY305310 |
| | | Roseburia intestinalis | AJ312385 |
| | Ruminococcus gnavus et rel. | Eubacterium contortum | L34615 |
| | | Ruminococcus gnavus | L76597 |
| | | Ruminococcus torques | L76604 |
| | | Clostridium oroticum | M59109 |
| | | Ruminococcus sp. CJ60 | AB080891 |
| | | uncultured human gut bacterium JW1H4a | AB080862 |
| | | uncultured bacterium (human infant) L37A | AF253389 |
| | | uncultured bacterium Adhufec117rbh | AY471716 |
| | | uncultured bacterium Muc2-3 | AY451997 |
| | Ruminococcus hansenii et rel. | Ruminococcus productus | D14144 |
| | | Clostridium coccoides | M59090 |
| | | Ruminococcus hansenii | M59114 |
| | | Ruminococcus hydrogenotrophicus | X95624 |
| | | uncultured bacterium KS62 | AY916137 |
| | Ruminococcus lactaris et rel. | bacterium adhufec80.25 | AF153858 |
| | | Ruminococcus lactaris | L76602 |
| | | uncultured bacterium G187 | AY916284 |
| | | uncultured bacterium L160 | AY916218 |
| | | uncultured bacterium HuRC19 | AY684372 |
| | Ruminococcus luti et rel. | butyrate-producing bacterium T2-132 | AJ270483 |
| | | uncultured Ruminococcus sp. NO3 | AB064755 |
| | | uncultured Ruminococcus sp. NB2F4 | AB064753 |
| | | uncultured Ruminococcus sp. NO2-22 | AB064751 |
| | | uncultured bacterium E177 | AY916259 |
| | | uncultured bacterium KS90 | AY916138 |
| | | uncultured bacterium L068 | AY916217 |
| | | uncultured bacterium HuCA5 | AJ408961 |
| | | Uncultured bacterium clone Eldhufec106 | AY919981 |
| | | Uncultured bacterium UC7-36 | AJ608238 |
| | | Uncultured bacterium UC7-7 | AJ608225 |
| | | Ruminococcus luti | AJ133124 |
| | | uncultured bacterium adhufec30.25 | AF153854 |
| | | uncultured bacterium Adhufec086abh | AY471687 |
| | | uncultured bacterium Adhufec048abh | AY471680 |
| | Ruminococcus obeum et rel. | bacterium adhufec35.25 | AF153853 |
| | | Ruminococcus obeum | L76601 |
| | | uncultured Ruminococcus sp. NO67 | AB064763 |
| | | uncultured bacterium KZ22 | AY916216 |
| | | uncultured bacterium NL49 | AY916245 |
| | | uncultured bacterium NQ96 | AY916255 |
| | | uncultured bacterium V127 | AY916274 |
| | | Uncultured bacterium UC7-35 | AJ608237 |
| | | uncultured bacterium Muc1-21 | AY451996 |
| | | uncultured bacterium Muc1-11 | AY451995 |
| | | uncultured bacterium Muc3-10 | AY452003 |
| | | uncultured bacterium Muc3-5 | AY452001 |
| | | uncultured bacterium Muc6-16 | AY452019 |
| | | uncultured bacterium Muc6-13 | AY452017 |
| | | bacterium ucfecDB7 | |
| | Unclutured Ruminococci | uncultured Ruminococcus sp. NS2E3 | AB064750 |
| | | uncultured human gut bacterium JW1B11 | AB080869 |
| | | uncultured human gut bacterium JW1H7 | AB080868 |
| | | uncultured bacterium K379 | AY916198 |
| | | uncultured bacterium ME10 | AY916229 |
| | | uncultured bacterium HuCB25 | AJ408999 |
| | | uncultured bacterium HuCA26 | AJ408977 |
| | | uncultured bacterium HuCA2 | AJ408958 |
| | | Uncultured bacterium clone Eldhufec132 | AY920007 |
| | | Uncultured bacterium clone Eldhufec133 | AY920008 |
| | | Uncultured bacterium clone Eldhufec102 | AY919977 |
| | | Uncultured bacterium UC7-23 | AJ608235 |
| | | uncultured bacterium cadhufec102c10 | AF530364 |
| | | uncultured bacterium cadhufec028h7 | AF530301 |
| | | uncultured bacterium A20 | AF052417 |
| | | uncultured bacterium A14 | AF052415 |
| | | uncultured bacterium HuDI20 | AY684379 |
| | | uncultured bacterium (human infant) L127 | AF253374 |
| | | uncultured bacterium (human infant) P36G | AF253346 |
| | | uncultured bacterium (human infant) P36H | AF253344 |
| | | uncultured bacterium Adhufec123khh | AY471668 |
| | | uncultured bacterium Muc3-9 | AY452002 |
| | | uncultured bacterium Muc4-13 | AY452010 |
| | | bacterium ucfecDB13 | |
| Clostridium | Eubacterium limosum et rel. | Pseudoramibacter alactolyticus | AB036759 |
| cluster XV | | Eubacterium limosum | AF064242 |
| | | Eubacterium barkeri | M23927 |
| | | Anaerofustis stercorihominis | AJ518871 |
| | | Eubacterium sp. CS1 Van | AJ518868 |
| Clostridium | Eubacterium biforme et rel. | uncultured bacterium D196 | AY916362 |
| cluster XVI | | Uncultured bacterium clone Eldhufec204 | AY920079 |
| | | Uncultured bacterium clone Eldhufec206 | AY920081 |
| | | butyrate-producing bacterium SM7/11 | AY305313 |
| | | Eubacterium biforme | M59230 |
| | | uncultured Gram-positive bacterium NB2C7 | AB064867 |
| | Eubacterium cylindroides et | Eubacterium cylindroides | L34616 |
| | rel. | Eubacterium dolichum | L34682 |
| | | Eubacterium tortuosum | L34683 |
| | | Clostridium innocuum | M23732 |
| | Solobacterium moorei et rel. | Holdemania filiformis | Y11466 |
| | | uncultured bacterium M615 | AY916164 |
| | | Uncultured bacterium clone Eldhufec205 | AY920080 |
| | | Solobacterium moorei | AY044916 |
| Clostridium | Catenibacterium | Lactobacillus vitulinus | M23727 |
| cluster XVII | | Lactobacillus catenaformis | M23729 |
| | | human intestinal firmicute CB12 | AB064934 |
| | | Uncultured bacterium clone Eldhufec203 | AY920078 |
| | | Catenibacterium mitsuokai | AB030226 |
| Clostridium | Clostridium ramosum et rel. | Clostridium cocleatum | AF028350 |
| cluster XVIII | | Clostridium ramosum | M23731 |
| | | Clostridium spiroforme | X73441 |
| | | Uncultured bacterium clone Eldhufec200 | AY920075 |
| | | Clostridium sp. 14774 | AJ315981 |
| | Coprobacillus catenaformis et | Coprobacillus catenaformis | AB030218 |
| | rel. | uncultured bacterium KU74 | AY916140 |
| | | uncultured bacterium NI20 | AY916175 |
| | | uncultured bacterium LCLC16 | AF499845 |
| Uncultured | Uncultured Clostridiales I | uncultured human gut bacterium JW2B4 | AB080852 |
| Clostridiales | | uncultured bacterium OLDA-F7 | AB099784 |
| | | uncultured bacterium OLDB-A9 | AB099783 |
| | | uncultured bacterium OLDCA-1 | AB099781 |
| | | uncultured bacterium C118 | AY916326 |
| | | uncultured bacterium C257 | AY916329 |
| | | uncultured bacterium C627 | AY916340 |
| | | uncultured bacterium D049 | AY916352 |
| | | uncultured bacterium D279 | AY916363 |
| | | uncultured bacterium D693 | AY916381 |
| | | uncultured bacterium LH65 | AY916208 |
| | | uncultured bacterium M220 | AY916150 |
| | | uncultured bacterium M233 | AY916151 |
| | | uncultured bacterium M412 | AY916156 |
| | | uncultured bacterium M621 | AY916165 |
| | | uncultured bacterium MF22 | AY916236 |
| | | uncultured bacterium MF35 | AY916239 |
| | | uncultured bacterium MG86 | AY916291 |
| | | uncultured bacterium NH06 | AY916173 |
| | | Uncultured bacterium clone Eldhufec312 | AY920187 |
| | | Uncultured bacterium clone Eldhufec309 | AY920184 |
| | | Uncultured bacterium clone Eldhufec311 | AY920186 |
| | | Uncultured bacterium clone Eldhufec308 | AY920183 |
| | | Uncultured bacterium clone Eldhufec310 | AY920185 |
| | | Uncultured bacterium clone Eldhufec314 | AY920189 |
| | | Uncultured bacterium UC7-9 | AJ608227 |
| | | Uncultured bacterium UC7-127 | AJ608249 |
| | Uncultured Clostridiales IIa | uncultured human gut bacterium JW2H12 | AB080880 |
| | | uncultured bacterium OLDB-C2 | AB099778 |
| | | uncultured bacterium C736 | AY916346 |
| | | uncultured bacterium LQ86 | AY916269 |
| | | uncultured bacterium M501 | AY916160 |
| | | Uncultured bacterium clone Eldhufec333 | AY920208 |
| | | Uncultured bacterium clone Eldhufec322 | AY920197 |
| | | Uncultured bacterium clone Eldhufec332 | AY920207 |
| | Uncultured Clostridiales IIb | uncultured human gut bacterium JW1H11 | AB080881 |
| | | uncultured human gut bacterium JW1B2 | AB080879 |
| | | uncultured bacterium OLDB-H1 | AB099779 |
| | | uncultured bacterium OLDB-F4 | AB099777 |
| | | uncultured bacterium C583 | AY916338 |
| | | uncultured bacterium C655 | AY916341 |
| | | uncultured bacterium D191 | AY916361 |
| | | uncultured bacterium K342 | AY916195 |
| | | uncultured bacterium M403 | AY916155 |
| | | uncultured bacterium MH87 | AY916298 |
| | | uncultured bacterium MM92 | AY916304 |
| | | uncultured bacterium HuCA6 | AJ408962 |
| | | Uncultured bacterium clone Eldhufec328 | AY920203 |
| | | Uncultured bacterium clone Eldhufec323 | AY920198 |
| | | Uncultured bacterium clone Eldhufec334 | AY920209 |
| | | Uncultured bacterium clone Eldhufec330 | AY920205 |
| | | Uncultured bacterium clone Eldhufec331 | AY920206 |
| | | Uncultured bacterium clone Eldhufec336 | AY920211 |
| | | Uncultured bacterium clone Eldhufec327 | AY920202 |
| | | Uncultured bacterium clone Eldhufec325 | AY920200 |
| | | Uncultured bacterium clone Eldhufec324 | AY920199 |
| | | Uncultured bacterium clone Eldhufec326 | AY920201 |
| | | uncultured bacterium cadhufec008h7 | AF530296 |
| | | uncultured bacterium cadhufec18c08 | AF530351 |
| | | uncultured bacterium cadhufec17f05 | AF530343 |
| | | uncultured bacterium Adhufec015rbh | AY471695 |
| | | uncultured bacterium Adhufec102abh | AY471690 |
| | | uncultured bacterium Adhufec123rbh | AY471719 |
| Uncultured | Uncultured Mollicutes | bacterium adhufec202 | AF132232 |
| Mollicutes | | bacterium adhufec279 | AF132233 |
| | | uncultured bacterium C027 | AY916325 |
| | | uncultured bacterium C133 | AY916328 |
| | | uncultured bacterium C611 | AY916339 |
| | | uncultured bacterium C754 | AY916348 |
| | | uncultured bacterium D051 | AY916353 |
| | | uncultured bacterium D423 | AY916369 |
| | | uncultured bacterium LW88 | AY916186 |
| | | uncultured bacterium MC12 | AY916226 |
| | | uncultured bacterium NB12 | AY916191 |
| | | Uncultured bacterium clone Eldhufec209 | AY920084 |
| | | Uncultured bacterium clone Eldhufec207 | AY920082 |
| | | Uncultured bacterium clone Eldhufec208 | AY920083 |
| Cyanobacteria | Uncultured Chroococcales | uncultured bacterium M019 | AY916143 |
| Fusobacteria | Cetobacterium | Cetobacterium somerae | AJ438155 |
| | Fusobacterium | Fusobacterium necrophorum | AF044948 |
| | | Fusobacterium naviforme | AJ006965 |
| | | Fusobacterium gonidoformans | M58679 |
| | | Fusobacterium mortiferum | M58680 |
| | | Fusobacterium varium | M58686 |
| | | Fusobacterium nucleatum | X55404 |
| | | Fusobacterium necrogenes | X55408 |
| | | Fusobacterium russii | X55409 |
| | | Clostridium rectum | X77850 |
| | | uncultured bacterium HuJJ10 | AY684429 |
| | Leptotrichia | Leptotrichia bucallis | L37788 |
| Alpha- | Methylobacterium | uncultured bacterium ABLCf14 | AF499910 |
| Proteobacteria | Novosphingobium | uncultured bacterium ABLCf85 | AF499911 |
| | Oceanospirillum | uncultured bacterium D623 | AY916377 |
| | | uncultured bacterium D784 | AY916388 |
| | | uncultured bacterium MK72 | AY916302 |
| | | uncultured bacterium V326 | AY916278 |
| Beta- | Alcaligenes faecalis et rel. | Achromobacter denitrificans | AF232712 |
| Proteobacteria | | uncultured bacterium ABLC15 | AF499888 |
| | | Alcaligenes faecalis | DQ110882 |
| | | Kerstersia gyiorum | AY131213 |
| | Aquabacterium | uncultured bacterium ABLC71 | AF499885 |
| | Burkholderia | uncultured bacterium LCLC40 | AF499842 |
| | Neisseria | uncultured bacterium HuJJ55 | AY684428 |
| | Oxalobacter formigenes et rel. | Oxalobacter formigenes | U49749 |
| | | uncultured bacterium ABLC55 | AF499887 |
| | Sutterella wadsworthia et rel. | Sutterella wadsworthia | L37785 |
| | | uncultured bacterium D093 | AY916355 |
| | | uncultured bacterium M105 | AY916147 |
| | | uncultured bacterium HuCA4 | AJ408960 |
| | | uncultured bacterium HuCC33 | AJ315485 |
| | | Uncultured bacterium clone Eldhufec064 | AY919939 |
| | | Uncultured bacterium clone Eldhufec063 | AY919938 |
| | | uncultured bacterium ABLC72 | AF499889 |
| | | uncultured bacterium HuDI12 | AY684426 |
| Gamma- | Aeromonas | Aeromonas veronii | AF099024 |
| Proteobacteria | | Aeromonas enteropelogenes | S42871 |
| | Anaerobiospirillum | Anaerobiospirillum thomasii | AJ420985 |
| | | Anaerobiospirillum succiniciproducens | U96412 |
| | Enterobacter aerogenes et rel. | Enterobacter aerogenes | AB004750 |
| | | Citrobacter freundii | AF025365 |
| | | Citrobacter koseri | AF025366 |
| | | Citrobacter braakii | AF025368 |
| | | Citrobacter werkmanii | AF025373 |
| | | Tatumella ptyseos | AJ233437 |
| | | Raoultella terrigena | Y17658 |
| | | Klebsiella oxytoca | Y17660 |
| | | Raoultella planticola | Y17663 |
| | | Enterobacter cancerogenus | Z96078 |
| | | uncultured bacterium OLDA-E9 | AB099791 |
| | | Citrobacter gillenii | AF025367 |
| | | Citrobacter murliniae | AF025369 |
| | | Averyella dalhousiensis | DQ481464 |
| | Escherichia coli et rel. | Escherichia coli | A14565 |
| | | Edwardsiella tarda | AF015259 |
| | | Citrobacter sedlakii | AF025364 |
| | | Citrobacter farmeri | AF025371 |
| | | Salmonella enterica | U90318 |
| | | Shigella flexneri | X80679 |
| | | Shigella dysenteriae | X80680 |
| | | Uncultured bacterium clone Eldhufec069 | AY919944 |
| | | Cedecea davisae | AF493976 |
| | | Escherichia fergusonii | AF530475 |
| | | Trabulsiella guamensis | AY373830 |
| | | Citrobacter amalonaticus | AF025370 |
| | | uncultured bacterium Muc4-17 | AY452011 |
| | Haemophilus | Haemophilus haemolyticus | M75045 |
| | | Haemophilus parainfluenzae | M75081 |
| | Klebsiella pneumoniae et rel. | Pantoea agglomerans | AB004691 |
| | | Serratia liquefaciens | AB004752 |
| | | Klebsiella pneumoniae | AB004753 |
| | | Enterobacter cloacae | AF157695 |
| | | Yokenella regensburgei | AY269192 |
| | | Enterobacter asburiae | AB004744 |
| | Leminorella | Leminorella grimontii | AJ233421 |
| | Moraxellaceae | Moraxella catarrhalis | A27627 |
| | | Acinetobacter calcoaceticus | AF159045 |
| | | Acinetobacter johnsonii | AF188300 |
| | | Acinetobacter haemolyticus | Z93437 |
| | | uncultured bacterium HuJJ26 | AY684425 |
| | | uncultured bacterium HuJJ19 | AY684423 |
| | Proteus et rel. | Providencia stuartii | AF008581 |
| | | Proteus mirabilis | AF008582 |
| | | Proteus vulgaris | AJ233425 |
| | | Morganella morganii | AJ301681 |
| | | Providencia alcalifaciens | AJ301684 |
| | | Providencia rettgeri | AM040492 |
| | | Providencia rustigianii | AM040489 |
| | | Moellerella wisconsensis | AM040754 |
| | | Proteus penneri | AJ634474 |
| | Pseudomonas | Pseudomonas aeruginosa | AB037545 |
| | | Pseudomonas stutzeri | AF038653 |
| | Pseuodomonas | Pseudomonas monteilii | AF064458 |
| | | Pseudomonas fluorescens | AJ278813 |
| | | Pseudomonas putida | D84020 |
| | Serratia | Serratia marcescens | M59160 |
| | Vibrio | Vibrio parahaemolyticus | M59161 |
| | | Grimontia hollisae | S83393 |
| | | Vibrio fluvialis | X74703 |
| | | Vibrio furnissii | X74704 |
| | Xanthomonadaceae | uncultured bacterium ABLCf21 | AF499898 |
| | | uncultured bacterium ABLC16 | AF499891 |
| | Yersinia et rel. | Yersinia pseudotuberculosis | AF282307 |
| | | Yersinia enterocolitica | AF282308 |
| | | Hafnia alvei | M59155 |
| | | Yersinia frederiksenii | X75273 |
| | | Yersinia rohdei | X75276 |
| | | Yersinia kristensenii | X75278 |
| | | Yersinia bercovieri | X75281 |
| Delta- | Bilophila | Bilophila wadsworthia | L35148 |
| Proteobacteria | Desulfovibrio et rel. | Desulfovibrio desulfuricans | AF098671 |
| | | Desulfvibrio piger | AF192152 |
| | | uncultured bacterium D168 | AY916359 |
| | | uncultured bacterium LE30 | AY916206 |
| | | Uncultured bacterium clone Eldhufec073 | AY919948 |
| | | Desulfovibrio fairfieldensis | U42221 |
| | | bacterium ucfecDB10 | |
| | | bacterium ucfecDB12 | |
| Epsilon- | Arcobacter | Arcobacter cryaerophilus | L14624 |
| Proteobacteria | | Arcobacter butzleri | U34386 |
| | Campylobacter | Campylobacter hominis | AF062490 |
| | | Campylobacter fetus | AJ306568 |
| | | Campylobacter jejuni | AL139074 |
| | | Campylobacter coli | L04312 |
| | | Campylobacter lari | L04316 |
| | | Campylobacter rectus | L04317 |
| | | Campylobacter gracilis | L04320 |
| | | Bacteroides ureolyticus | L04321 |
| | | Campylobacter concisus | L04322 |
| | | Campylobacter upsaliensis | L14628 |
| | Helicobacter | Helicobacter pylori | AE000511 |
| | | Flexispira rappini | AF034135 |
| | | Helicobacter canadensis | AF262037 |
| | | Helicobacter cinaedi | AF396082 |
| | | Helicobacter pullorum | L36141 |
| | | Helicobacter winghamensis | AF246984 |
| Lentisphaerae | Victivallis | Victivallis vadensis | AY049713 |
| Spirochaetes | Brachyspira | Brachyspira aalborgi | AF395882 |
| | | Brachyspira pilosicoli | AY155458 |
| Verruco-microbia | Akkermansia | Uncultured bacterium clone Eldhufec002 | AY919877 |
| | | Akkermansia muciniphila | AY271254 |
| | | uncultured bacterium HuRC51 | AY684431 |

### EXAMPLES

### Example 1

### Comparison of the Fecal Microbiota of IBS and Healthy Subjects (Study 1)

Fecal samples were obtained from a first study (Study 1) of a total of 62 IBS subjects including 19 with IBS-C, 25 with IBS-D and 18 with IBS-A, and a total of 46 healthy individuals that were age and gender matched. Microbial DNA was isolated from these fecal samples following the method of Ahlroos & Tynkynnen (2009, supra) and used for profiling using the HITChip phylogenetic microarray using 3699 distinct HIT probes as described (Rajilic-Stojanovic et al., 2009, supra). Based on the intenstity of the hybridization signals obtained in the HITChip analysis from the 62 IBS subjects and 46 healthy individuals a total of 36 level 2 microbial groups from the total of over 100 groups was found to be reacting significantly different between IBS and healthy subjects (see Table 1 above). The identified microbial groups can be developed as biomarker as described above. Moreover, the differences in microbiota can be corrected to the healthy level. This can be directly realized by consuming the microbes and/or their proteins or metabolites that are reduced in the IBS subjects, as if they were probiotics. This has already been suggested for Faecalibacterium prauznitzii in the case of IBD and here we extend this approach for said bacteria to the case of IBS (Sokol et al., 2008. Proc Natl Acad Sci USA 105: 16731-36). In addition, indirect modulation of the presence or absence of specific microbial groups can also be realized by the consumption of pre- and probiotics or its combination. Lastly, for the in the invention identified microbiota that are related to bioactive pathways, these pathways too can be used or targeted for the treatment of IBS.

### Example 2

### Identification of IBS- and Healthy-Specific Oligonucleotides

In order to further define the specific oligonucleotide probes that were reacting different in the IBS subjects as compared to the healthy controls, the hybridization of all 3,699 HIT probes of the HITChip in Study 1 (Example 1) was analyzed, resulting in a total of 100 HIT probes were found to be differentially hybridizing (Tables 2 and 4). A total of 34 HIT probes (oligonucleotides having SEQ ID Nos:1-27, 70-71, 73-77, 99-100) showed a significantly higher hybridization signal in the IBS subjects than the healthy individuals, while a total of 66 (oligonucleotides having SEQ ID Nos:28-69, 72, 78-98) showed less hybridization in the IBS subjects than the healthy subjects, respectively. The sequences of these oligonucleotides are disclosed in Tables 2 and 4 and allow the development of specific probes as described above. Moreover, these probes can be used to screen the 16S rDNA databases for complete 16S rRNA sequences that subsequently can be used as target for the development of specific probes as described above. This has been done using the SILVA and RDP databases using the ProbeCheck program (http://131.130.66.200/cgi-bin/probecheck/probecheck.pl). As the discriminating oligonucleotides are used in a hybridization assay, their complementarity to a 16S rRNA gene should not necessarily be perfect and mismatches up to 2 nucleotides can be envisaged. Hence the SILVA and RDP databases were searched for 16S rRNA gene sequences using the discriminating IBS- and Health-specific oligonucleotides allowing up to 2 mismatches. This resulted in multiple hits for each of the oligonucleotides showing the feasibility of this approach.

### Example 3

### Further Analysis of the Differences in Fecal Microbiota of IBS and Healthy Subjects

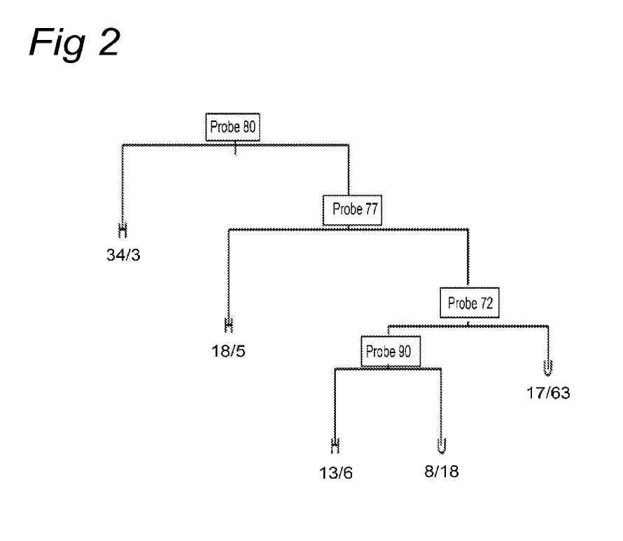

To further substantiate the differentiation of IBS subjects and healthy controls based on fecal microbiota, a second set of samples was analyzed that included a total of 33 IBS subjects that were not further differentiated and 43 healthy controls that were age and gender matched (Study 2). Fecal samples were obtained from these 77 individuals and microbial DNA was isolated from these following the repeated bead beating method as described (Yu & Morrison, 2004, supra). This DNA was used for profiling using the HITChip phylogenetic microarray using 3699 distinct HIT probes as described (Rajilic-Stojanovic et al., 2009, supra). As the DNA extraction method differed between Study 1 (Example 1) and Study 2 (the results presented here) as an enzymatic and mechanical lysis method was used, respectively, it was of interest to see the differentiation of the datasets obtained from the HITChip analysis in both tests. A Redundancy Analysis (RDA) was performed using all data from both Study 1 and Study 2. The results (FIG. 2) show a remarkable separation between samples from IBS subjects and healthy controls.

This indicates that in spite of being derived from 2 different studies and 2 different DNA extraction methods, the obtained data sets are sufficiently robust to show a clear separation between IBS subjects and healthy controls. Moreover, this analysis demonstrates that it is possible to differentiate IBS subjects from health controls based on biomarkers derived from their intestinal microbiota.

### Example 4

### Detection and Benchmarking Diagnostic Probes

To further detect and benchmark specific HIT probes that were potential diagnostic markers to differentiate between fecal microbiota of IBS subjects and healthy controls, the data sets obtained from Study 1 and Study 2 were combined. Subsequently, a training data set, consisting of ⅔ of the data, and a test data set, consisting of ⅓ of the data, were randomly selected. The rationale behind this division of the data sets is that the test data are not used at all in the modeling or selection process but only in the final testing. This should protect from over-fitting of the models into the data (i.e. from an inferior generalization). The training data was used to filter out the most discriminating HIT probes using a t-test. These are listed in Table 3. They were used to classify the training set with different classifiers, including stepwise linear discriminant analysis (LDA), a multivariant analysis system (see Venables, W. N. and Ripley, B. D. (2002) Modern Applied Statistics with S. Fourth edition. Springer Publishers). The subsequent classification was done in two nested cross-validation loops, where the inner one was used to select the discriminating features in a stepwise-LDA, and the outer loop to validate the performance of the classifiers for unseen data. The final test simulation was done by applying the stepwise-LDA to all of the training data, and then classifying the ⅓ of the binded test data, and comparing it to the 10 randomized classifications. A clinically meaningful separation could be obtained that When this stepwise LDA was applied to the ⅓ of the blinded test data, a correct classification was realized of 81% of the samples derived from the IBS subjects. When the obtained result was compared to the randomized classifications (repeated 10 times) using t-test, the difference between the non-randomized classification and the randomized classifications was found to be statistically highly significant (p-value 6.697e-09). This result was obtained with the HIT probes with the SEQ ID No 83 and 88 (Table 4). Hence, this example shows that a clinically meaningful diagnosis could be already realized with the the lowest number of multiple HIT probes, namely two probes.

TABLE 4
Identification, sequence and analysis of the
HIT probes coded SEQ ID 68-100 that were ob-
tained in the stepwise linear discriminant
analysis of various parts of the datasets of
Study 1 and Study 2. The oligonucleotides
are indicated with their nucleotide sequence
(3′ to 5′). The oligonucleotides with SEQ ID
Nos: 70-71, 73-77, 99-100 showed a signifi-
cantly higher hybridization signal in the
IBS than the healthy subjects, whereas the
oligonucleotides with SEQ ID Nos: 68-69, 72,
78-98 showed the opposite.

| | SEQ |
|---|---|
| sequence 5′ to 3′ | ID |
| direction (T = U in RNA) | NO: |
| CACCCCTCCTTTTCGGGAG | 68 |
| TAAACTACTTCCCGCTGCCGC | 69 |
| GCCGCTAATCCACTTCCCGAA | 70 |
| TGTCTCATTACGAGCAAGCTCACG | 71 |
| GGTCACTCGATGTCAAGACCTG | 72 |
| GTCAAAGGAGCAAGCTCCTCG | 73 |
| TACGTCACTCGATGTCAAGACCTG | 74 |
| TTCGTCACTCGATGTCAAGACCTG | 75 |
| AACGTCACTCGATGTCAAGACCTG | 76 |
| GCCACTCAGTCATAAAAAACTTCATC | 77 |
| GCCACTCAGTCATAAAAAACTTCATTC | 78 |
| GCCGCTCAGTCACTTAAGAAATCA | 79 |
| CGAAGTCCGTGCTGCCG | 80 |
| GCCGCTCAGTCACAAAGACTTCAA | 81 |
| AAATCCATCCGAAAACTTCATTTTAATTGC | 82 |
| GCCACTCGCCACCAGACC | 83 |
| TGTCTCCTCTGTCCGTAGAAAAAA | 84 |
| GCCGGTCGCCATCTTTAGTTTG | 85 |
| CAAGCTCCCTTTGGTCCGC | 86 |
| TGTCACTCTGCTCCCGAAGGA | 87 |
| TGTCTCTCTGTTCCCGAAGGAAA | 88 |
| TGTCTTCCTGCCCCGAAGC | 89 |
| GACATCATGCACCTCTGCACTATG | 90 |
| GCCACTCGTCACCGAAGGA | 91 |
| AGCAAGCTCCCTTCATCCGC | 92 |
| CACCGCCTCATCTCCGAG | 93 |
| GCCACTCGCCACCAGGTG | 94 |
| TGTCTCTCTGTTCCCGAAGGAAAC | 95 |
| TGTCACTCTGTTCCCGAAGAAC | 96 |
| GCCACCCAGTCACTTGAGC | 97 |
| CCACTCGCCACCAGGG | 98 |
| CCGCCAGGATTGCTCCCG | 99 |
| TGTCTCGTATTGAGCAAGCTCACA | 100 |

### Example 5

To further substantiate that combinations of HIT probes can be used in a diagnostic test to differentiate IBS subjects from healthy controls using all 185 subjects derived from Study 1 and Study 2, a number of these were analysed in a hierarchical analysis. The power of combining four discriminating HIT probes could be easily illustrated in a hierarchial decision tree (FIG. 2). It could be shown that hybridization to HIT probe with ID Seq 80 and its cut off at a certain hybridization value allowed to assign correctly 34 of healthy controls as healthy and 3 IBS subjects falsely. Similarly, a second HIT probe with ID Seq 77 could be used for further differentiating the remaining 148 subjects and could assign 18 healthy controls correctly and 5 IBS ones falsely. Subsequently, a third HIT probe with ID Seq 72 could be used to differentiate the remaining 125 subjects and could assign 63 IBS subjects correctly and 17 healthy controls incorrectly. Finally, ID Seq 90 could be applied to differentiate the remaining 45 subjects and this resulted in the correct assignment of 13 Healthy controls and 18 IBS subjects, while 6 IBS subjects and 8 healthy controls were falsely assigned. Altogether the use of these 4 HIT probes resulted in the correct classification of 85% of the IBS subjects. For those experienced in the art it will be evident that a strict classification can be obtained by using combinations of several of the HIT probes in conjunction with different cut-off values.

The probes that added significant value to the first classification (FIG. 2) were the probes 72, 77 and 90 that are specific for the bacterial taxa including Eubacterium sireaeum et rel., Lachnospira pectinoschiza et rel. and Subdoligranulum variabile et rel., respectively. These bacterial taxa already had been identified in a separate analysis when addressing Study 1 (see Table 1). This result testifies for the power of diagnosing IBS by determining the level of various and different groups of IBS-increased or IBS-decreased bacteria and using these in a decision tree as described here.

1. A method for diagnosing and/or subtyping Irritable Bowel Syndrome (IBS) in a test sample, said method comprising the steps of:
a) determining the levels of two or more bacteria which are present in statistically significantly different levels between IBS subjects and healthy subjects, said bacteria being selected from IBS-decreased bacteria and IBS-increased bacteria, said IBS-decreased bacteria being selected from bacteria belonging to the supertaxon Bacteroidetes, selected from the taxa Prevotella melaninogenica et rel., Prevotella oralis et rel., Uncultured Bacteroidetes, Tannerella et rel., Parabacteroides distasonis et rel., Allistipes et rel., Bacteroides plebeius et rel., Bacteroides splachnicus et rel., or to the supertaxon Clostridium cluster IV, selected from the taxa Subdoligranulum variabile et rel., Faecalibacterium prausnitzii et rel., Oscillospira guillermondii et rel., Sporobacter termitidis et rel., Ruminococcus callidus et rel., Eubacterium siraeum et rel., Anaerotruncus colihominis et rel., Clostridium cellulosi et rel., Clostridium leptum et rel., Ruminococcus bromii et rel., or to the supertaxon Clostridium cluster IX, said bacteria belonging to the taxon Phascolarctobacterium faecium et rel.; or to the supertaxon Clostridium cluster XVI, said bacteria belonging to the taxon Eubacterium biforme et rel.; or to the supertaxon Clostridium cluster XVII, said bacteria belonging to the taxon Catenibacterium mitsuokai et rel.; or to the supertaxon Proteobacteria, said bacteria belonging to the taxon Xanthomonadaceae; or to the supertaxon Uncultured Clostridiales, selected from the taxa Uncultured Clostridiales I and Uncultured Clostridiales II; or to the supertaxon Uncultured Mollicutes, said bacteria belonging to the taxon Uncultured Mollicutes, and said IBS-increased bacteria being selected from bacteria belonging to the supertaxon Clostridium cluster XIVa, selected from the taxa Dorea formicigenerans et rel., Ruminococcus obeum et rel., Clostridium nexile et rel., Clostridium symbiosum et rel., Outgrouping Clostridium cluster XIVa, Ruminococcus lactaris et rel., Lachnospira pectinoschiza et rel.; in a test sample;
b) comparing said level of said two or more IBS-decreased and/or IBS-increased bacteria in said test sample to a level of said two or more IBS-decreased and/or IBS-increased bacteria in a control sample; and
c1) relating a decreased level of said IBS-decreased bacteria and/or an increased level of said IBS-increased bacteria in the test sample compared to the control sample to a diagnosis that the test sample is from a subject suffering from Irritable Bowel Syndrome; and/or
c2) relating an increased level of said IBS-increased bacteria or a decreased level of said IBS-decreased bacteria in the test sample compared to the control sample to a diagnosis of whether the test sample is from a subject suffering from IBS-A, IBS-C, or IBS-D.

2. A method according to claim 1, wherein in step a) the levels of at least one IBS-increased bacteria and at least one IBS-decreased bacteria are determined.

3. A method according to claim 2, wherein in step a) the level of at least one IBS-increased bacteria selected from bacteria belonging to the taxa Dorea formicigenerans et rel., Ruminococcus obeum et rel., and Lachnospira pectinoschiza et rel., and the level of at least one IBS-decreased bacteria selected from bacteria belonging to the taxa Prevotella melaninogenica et rel, Prevotella oralis et rel., and Catenibacterium mitsuokai et rel., are determined.

4. A method according to claim 3, wherein in step a) at least the level of bacteria belonging to the taxa Dorea formicigenerans et rel., Ruminococcus obeum et rel., and Lachnospira pectinoschiza et rel., and the level of bacteria belonging to the taxa Prevotella melaninogenica et rel, Prevotella oralis et rel., and Catenibacterium mitsuokai et rel., are determined.

5. A method according to claim 1, wherein the level of said one or more bacteria is measured by determining the level of nucleic acid sequences, amino acid sequences and/or metabolites specific for said one or more bacteria in said test sample.

6. A method according to claim 5, wherein the level of nucleic acid sequences specific for said one or more bacteria are determined using PCR or LCR.

7. A method for diagnosing and/or subtyping Irritable Bowel Syndrome (IBS) in a test sample, said method comprising the steps of:
i) providing a test sample;
ii) determining the level of at least three nucleic acids capable of hybridising to at least three nucleic acid sequences selected from the nucleic acid sequences of SEQ ID Nos:1-100, or derivatives or fragments thereof deviating by at most 2 nucleotides, and complements, reverse, and reverse complements thereof, under stringent hybridization conditions, in said test sample;
ii) comparing the level of said at least three nucleic acids from said test sample to the level of said at least three nucleic acids from a control sample; and
iiia) relating the level of said at least three nucleic acids from said test sample to a diagnosis of whether the test sample is from a subject suffering from Irritable Bowel Syndrome; and/or
iiib) relating the level of said at least three nucleic acids from said test sample to a diagnosis of whether the test sample is from a subject suffering from IBS-A, IBS-C, or IBS-D.

8. A method according to claim 7, wherein in step iiia) an increased level of nucleic acids from said test sample, said nucleic acids being capable of hybridising to nucleic acid sequences selected from the nucleic acid sequences of SEQ ID Nos:1-27, 70-71, 73-77, 99-100, or derivatives or fragments thereof deviating by at most 2 nucleotides, and complements, reverse, and reverse complements thereof, under stringent hybridization conditions, compared to the level of said nucleic acids from said control sample relates to the diagnosis that the subject is suffering from IBS.

9. A method according to claim 7, wherein in step iiia) a decreased level of nucleic acids from said test sample, said nucleic acids being capable of hybridising to nucleic acid sequences selected from the nucleic acid sequences of SEQ ID Nos:28-69, 72, 78-98, or derivatives or fragments thereof deviating by at most 2 nucleotides, and complements, reverse, and reverse complements thereof, under stringent hybridization conditions, compared to the level of said nucleic acids from said control sample relates to the diagnosis that the subject is suffering from IBS.

10. A method according to claim 7, wherein the level of at least 6 nucleic acid sequences from said test sample is determined.

11. A method according to claim 7, wherein Significance Analysis of Microarrays (SAM) is used in comparing the levels of said three or more nucleic acid sequence from said test sample with the levels of said three or more nucleic acid sequence from a control sample.

12. A method according to claim 7, wherein Prediction Analysis of Microarray (PAM) is used in comparing the levels of said three or more nucleic acid sequence from said test sample with the levels of said three or more nucleic acid sequence from a control sample.

13. A method according to claim 7, wherein Redundancy Analysis is used in comparing the levels of said three or more nucleic acid sequence from said test sample with the levels of said three or more nucleic acid sequence from a control sample.

14. A method for diagnosing and/or subtyping Irritable Bowel Syndrome (IBS) in a test sample, said method comprising the steps of:
i) providing a test sample;
ii) determining the level of at least three nucleic acids capable of hybridising to 16S rRNA nucleic acid sequences hybridizing to the complementary strand of any of the nucleic acid sequences SEQ ID NO.:1-100 or fragments of said 16S rRNA nucleic acid sequences hybridizing to the complementary strand of any of the nucleic acid sequences SEQ ID NO.:1-100, and complements, reverse, and reverse complements thereof, under stringent hybridization conditions, in said test sample;
ii) comparing the level of said at least three nucleic acids from said test sample to the level of said at least three nucleic acids from a control sample; and
iiia) relating the level of said at least three nucleic acids from said test sample to a diagnosis of whether the test sample is from a subject suffering from Irritable Bowel Syndrome; and/or
iiib) relating the level of said at least three nucleic acids from said test sample to a diagnosis of whether the test sample is from a subject suffering from IBS-A, IBS-C, or IBS-D.

15. A method according to claim 7, wherein the level is determined using a method selected from: hybridization of the nucleic acids in a sample to the nucleic acid sequences having SEQ ID NO.:1-100, and complements, reverse, and reverse complements thereof, under stringent hybridization conditions; a Polymerase Chain reaction (PCR) or a Ligase Chain Reaction (LCR).

16. An array for diagnosing IBS and/or subtyping IBS-A, IBS-C, or IBS-D, said array comprising at least two nucleic acid sequences having the nucleic acid sequence of SEQ ID NOs: 1-100, or derivatives or fragments thereof deviating by at most 2 nucleotides, or complements, reverse, and reverse complements thereof.

17. An array according to claim 16, which comprises at least two nucleic acid sequences selected from the nucleic acid sequences having SEQ ID Nos:1-100.

18. An array according to claim 16, wherein the at least two nucleic acid sequences are bound to a solid phase matrix.

19. An array according to claim 16, wherein the array is a DNA or RNA array.

20. An array according to claim 16, which is a micro-array.

21. Use of an array according to claim 16 for diagnosing IBS and/or subtyping IBS-A, IBS-C, or IBS-D.

22. A method according to claim 14, wherein the level is determined using a method selected from: hybridization of nucleic acids in a sample to the nucleic acid sequences having SEQ ID NO.:1-100, and complements, reverse, and reverse complements thereof, under stringent hybridization conditions; a Polymerase Chain reaction (PCR) or Ligase Chain Reaction (LCR).


SEQUENCE LISTING (100 sequences)

SEQ ID NO: 1 - DNA, 20 bp, Artificial sequence
  Probe 1
gccgctcagtcacaatcctc

SEQ ID NO: 2 - DNA, 25 bp, Artificial sequence
  Probe 2
gccactagaaatagatcaaatccac

SEQ ID NO: 3 - DNA, 24 bp, Artificial sequence
  Probe 3
gccgctcagtcacaaaactcttca

SEQ ID NO: 4 - DNA, 26 bp, Artificial sequence
  Probe 4
ccgaagtttcaataaagtaattcccg

SEQ ID NO: 5 - DNA, 27 bp, Artificial sequence
  Probe 5
gccactagaattaaattaaatcgaccg

SEQ ID NO: 6 - DNA, 25 bp, Artificial sequence
  Probe 6
cgaagtctcaatgaaatatttcccg

SEQ ID NO: 7 - DNA, 25 bp, Artificial sequence
  Probe 7
cactagaaatagatcaaatccaccg

SEQ ID NO: 8 - DNA, 21 bp, Artificial sequence
  Probe 8
gccactcagtcacagtctctc

SEQ ID NO: 9 - DNA, 18 bp, Artificial sequence
  Probe 9
gccgctcagtcaccaagg

SEQ ID NO: 10 - DNA, 20 bp, Artificial sequence
  Probe 10
gccgctcagtcacaacactc

SEQ ID NO: 11 - DNA, 19 bp, Artificial sequence
  Probe 11
gccgctcagtcacaaaacc

SEQ ID NO: 12 - DNA, 20 bp, Artificial sequence
  Probe 12
gccgctcagtcacaaacgga

SEQ ID NO: 13 - DNA, 18 bp, Artificial sequence
  Probe 13
gccgctcagtcactgtcc

SEQ ID NO: 14 - DNA, 27 bp, Artificial sequence
  Probe 14
gccactagaattaaattatatcgaccg

SEQ ID NO: 15 - DNA, 26 bp, Artificial sequence
  Probe 15
gccactagaattaaatcatatcgacc

SEQ ID NO: 16 - DNA, 18 bp, Artificial sequence
  Probe 16
tgtctccgctgccccgaa

SEQ ID NO: 17 - DNA, 30 bp, Artificial sequence
  Probe 17
taaatcatatcgaccgaagtttcaataaaa

SEQ ID NO: 18 - DNA, 29 bp, Artificial sequence
  Probe 18
aaattatatcgaccgaagtttcaataaag

SEQ ID NO: 19 - DNA, 26 bp, Artificial sequence
  Probe 19
gccactagaaataaatcaaatccacc

SEQ ID NO: 20 - DNA, 20 bp, Artificial sequence
  Probe 20
agcaagctcctccttcagcg

SEQ ID NO: 21 - DNA, 25 bp, Artificial sequence
  Probe 21
atcctcttcatccgaagaatctaag

SEQ ID NO: 22 - DNA, 24 bp, Artificial sequence
  Probe 22
gccgctttccactcttaacttcaa

SEQ ID NO: 23 - DNA, 23 bp, Artificial sequence
  Probe 23
agaaatccgtcaaggtgcttcgc

SEQ ID NO: 24 - DNA, 28 bp, Artificial sequence
  Probe 24
gaagtttcaataaaataattcccgttcg

SEQ ID NO: 25 - DNA, 24 bp, Artificial sequence
  Probe 25
tgtcctcttcctccgaagattctg

SEQ ID NO: 26 - DNA, 26 bp, Artificial sequence
  Probe 26
ccgaagtttcaataaaataattcccg

SEQ ID NO: 27 - DNA, 24 bp, Artificial sequence
  Probe 27
gatccgtttaaggtgcttcgttcg

SEQ ID NO: 28 - DNA, 23 bp, Artificial sequence
  Probe 28
tgtctctgcgtcccgaaggaaaa

SEQ ID NO: 29 - DNA, 23 bp, Artificial sequence
  Probe 29
tgtctctgcgtcccgaaggaata

SEQ ID NO: 30 - DNA, 22 bp, Artificial sequence
  Probe 30
tgtctctgcgtcccgaaggaaa

SEQ ID NO: 31 - DNA, 20 bp, Artificial sequence
  Probe 31
gccactgtcctctgcttcac

SEQ ID NO: 32 - DNA, 24 bp, Artificial sequence
  Probe 32
atcgtcgcaggatgtcaagacttg

SEQ ID NO: 33 - DNA, 19 bp, Artificial sequence
  Probe 33
caagctcctctcagctccg

SEQ ID NO: 34 - DNA, 24 bp, Artificial sequence
  Probe 34
ggctgacatgtctccacatcattc

SEQ ID NO: 35 - DNA, 22 bp, Artificial sequence
  Probe 35
cgtcgcaggatgtcaagacttg

SEQ ID NO: 36 - DNA, 21 bp, Artificial sequence
  Probe 36
accgtcgcaggatgtcaagac

SEQ ID NO: 37 - DNA, 23 bp, Artificial sequence
  Probe 37
tgtctctgctgtcccgaaggaaa

SEQ ID NO: 38 - DNA, 21 bp, Artificial sequence
  Probe 38
gccactgtcctctgcttcgaa

SEQ ID NO: 39 - DNA, 24 bp, Artificial sequence
  Probe 39
atcgtcaagggatgtcaagacttg

SEQ ID NO: 40 - DNA, 21 bp, Artificial sequence
  Probe 40
tgcgtcgcaggatgtcaagac

SEQ ID NO: 41 - DNA, 24 bp, Artificial sequence
  Probe 41
cattcagttgcaattcaagcccgg

SEQ ID NO: 42 - DNA, 23 bp, Artificial sequence
  Probe 42
gccactttcctctacatccattg

SEQ ID NO: 43 - DNA, 23 bp, Artificial sequence
  Probe 43
ggatttcacacatctctgtgcta

SEQ ID NO: 44 - DNA, 24 bp, Artificial sequence
  Probe 44
ttcgtcaagggatgtcaagacttg

SEQ ID NO: 45 - DNA, 22 bp, Artificial sequence
  Probe 45
gttcgtcaagggatgtcaagac

SEQ ID NO: 46 - DNA, 23 bp, Artificial sequence
  Probe 46
gccactcgatttgaagagcaagc

SEQ ID NO: 47 - DNA, 24 bp, Artificial sequence
  Probe 47
gccactaaccgctccaatagtaaa

SEQ ID NO: 48 - DNA, 24 bp, Artificial sequence
  Probe 48
gatttgaagagcaagctcctcatc

SEQ ID NO: 49 - DNA, 22 bp, Artificial sequence
  Probe 49
gccactcgatcaaggaagcaag

SEQ ID NO: 50 - DNA, 22 bp, Artificial sequence
  Probe 50
ttcacaactgccttgcggctga

SEQ ID NO: 51 - DNA, 24 bp, Artificial sequence
  Probe 51
cctctttccacagattctcgttcg

SEQ ID NO: 52 - DNA, 23 bp, Artificial sequence
  Probe 52
cgatttgaagagcaagctcctca

SEQ ID NO: 53 - DNA, 24 bp, Artificial sequence
  Probe 53
gaatccgtaatcaagcttcgttcg

SEQ ID NO: 54 - DNA, 22 bp, Artificial sequence
  Probe 54
ttctcctgcaattcaagcccgg

SEQ ID NO: 55 - DNA, 24 bp, Artificial sequence
  Probe 55
tcgttagcaggatgtcaaaccctg

SEQ ID NO: 56 - DNA, 22 bp, Artificial sequence
  Probe 56
atgcacctgcaattcaagcccg

SEQ ID NO: 57 - DNA, 22 bp, Artificial sequence
  Probe 57
caagctcctcatctctcgttcg

SEQ ID NO: 58 - DNA, 23 bp, Artificial sequence
  Probe 58
tgtctccttgctccgaagagaaa

SEQ ID NO: 59 - DNA, 24 bp, Artificial sequence
  Probe 59
tgtctccttgctccgaagagaaaa

SEQ ID NO: 60 - DNA, 24 bp, Artificial sequence
  Probe 60
tgtctccttgctccgaagagatta

SEQ ID NO: 61 - DNA, 24 bp, Artificial sequence
  Probe 61
tgtctcgatgtcccgaaggatttc

SEQ ID NO: 62 - DNA, 22 bp, Artificial sequence
  Probe 62
agagcaagctcctcatctctcg

SEQ ID NO: 63 - DNA, 25 bp, Artificial sequence
  Probe 63
gccactagattgtagaaaaagcaag

SEQ ID NO: 64 - DNA, 23 bp, Artificial sequence
  Probe 64
gcacctaatgcatctctgcttcg

SEQ ID NO: 65 - DNA, 22 bp, Artificial sequence
  Probe 65
gaagcaagcttcctctctctcg

SEQ ID NO: 66 - DNA, 23 bp, Artificial sequence
  Probe 66
caagctcctcttgattccgttcg

SEQ ID NO: 67 - DNA, 27 bp, Artificial sequence
  Probe 67
agagaattattagcaagctagcaattc

SEQ ID NO: 68 - DNA, 19 bp, Artificial sequence
  Probe 68
cacccctccttttcgggag

SEQ ID NO: 69 - DNA, 21 bp, Artificial sequence
  Probe 69
taaactacttcccgctgccgc

SEQ ID NO: 70 - DNA, 21 bp, Artificial sequence
  Probe 70
gccgctaatccacttcccgaa

SEQ ID NO: 71 - DNA, 24 bp, Artificial sequence
  Probe 71
tgtctcattacgagcaagctcacg

SEQ ID NO: 72 - DNA, 22 bp, Artificial sequence
  Probe 72
ggtcactcgatgtcaagacctg

SEQ ID NO: 73 - DNA, 21 bp, Artificial sequence
  Probe 73
gtcaaaggagcaagctcctcg

SEQ ID NO: 74 - DNA, 24 bp, Artificial sequence
  Probe 74
tacgtcactcgatgtcaagacctg

SEQ ID NO: 75 - DNA, 24 bp, Artificial sequence
  Probe 75
ttcgtcactcgatgtcaagacctg

SEQ ID NO: 76 - DNA, 24 bp, Artificial sequence
  Probe 76
aacgtcactcgatgtcaagacctg

SEQ ID NO: 77 - DNA, 26 bp, Artificial sequence
  Probe 77
gccactcagtcataaaaaacttcatc

SEQ ID NO: 78 - DNA, 27 bp, Artificial sequence
  Probe 78
gccactcagtcataaaaaacttcattc

SEQ ID NO: 79 - DNA, 24 bp, Artificial sequence
  Probe 79
gccgctcagtcacttaagaaatca

SEQ ID NO: 80 - DNA, 17 bp, Artificial sequence
  Probe 80
cgaagtccgtgctgccg

SEQ ID NO: 81 - DNA, 24 bp, Artificial sequence
  Probe 81
gccgctcagtcacaaagacttcaa

SEQ ID NO: 82 - DNA, 30 bp, Artificial sequence
  Probe 82
aaatccatccgaaaacttcattttaattgc

SEQ ID NO: 83 - DNA, 18 bp, Artificial sequence
  Probe 83
gccactcgccaccagacc

SEQ ID NO: 84 - DNA, 24 bp, Artificial sequence
  Probe 84
tgtctcctctgtccgtagaaaaaa

SEQ ID NO: 85 - DNA, 22 bp, Artificial sequence
  Probe 85
gccggtcgccatctttagtttg

SEQ ID NO: 86 - DNA, 19 bp, Artificial sequence
  Probe 86
caagctccctttggtccgc

SEQ ID NO: 87 - DNA, 21 bp, Artificial sequence
  Probe 87
tgtcactctgctcccgaagga

SEQ ID NO: 88 - DNA, 23 bp, Artificial sequence
  Probe 88
tgtctctctgttcccgaaggaaa

SEQ ID NO: 89 - DNA, 19 bp, Artificial sequence
  Probe 89
tgtcttcctgccccgaagc

SEQ ID NO: 90 - DNA, 24 bp, Artificial sequence
  Probe 90
gacatcatgcacctctgcactatg

SEQ ID NO: 91 - DNA, 19 bp, Artificial sequence
  Probe 91
gccactcgtcaccgaagga

SEQ ID NO: 92 - DNA, 20 bp, Artificial sequence
  Probe 92
agcaagctcccttcatccgc

SEQ ID NO: 93 - DNA, 18 bp, Artificial sequence
  Probe 93
caccgcctcatctccgag

SEQ ID NO: 94 - DNA, 18 bp, Artificial sequence
  Probe 94
gccactcgccaccaggtg

SEQ ID NO: 95 - DNA, 24 bp, Artificial sequence
  Probe 95
tgtctctctgttcccgaaggaaac

SEQ ID NO: 96 - DNA, 22 bp, Artificial sequence
  Probe 96
tgtcactctgttcccgaagaac

SEQ ID NO: 97 - DNA, 19 bp, Artificial sequence
  Probe 97
gccacccagtcacttgagc

SEQ ID NO: 98 - DNA, 16 bp, Artificial sequence
  Probe 98
ccactcgccaccaggg

SEQ ID NO: 99 - DNA, 18 bp, Artificial sequence
  Probe 99
ccgccaggattgctcccg

SEQ ID NO: 100 - DNA, 24 bp, Artificial sequence
  Probe 100
tgtctcgtattgagcaagctcaca